(19) United States
(12) Patent Application Publication
(10) Pub. No.: US 2007/0219224 A1
(43) Pub. Date: Sep. 20, 2007

(54) Compositions and Methods Relating to Pyrimidine Synthesis Inhibitors

(75) Inventors: Sadis Matalon, Mountain Brook, AL (US); Ian C. Davis, Hilliard, OH (US)

Correspondence Address: FISH & RICHARDSON P.C., P.O BOX 1022, Minneapolis, MN, 55440-1022, US

(73) Assignee: UAB RESEARCH FOUNDATION, THE, Birmingham, AL (US)

(21) Appl. No.: 11/569,316

(22) PCT Filed: May 20, 2005
(86) PCT No.: PCT/US05/17939

§ 371 Date: May 2, 2007

Related U.S. Application Data
(60) Provisional application No. 60/573,558, filed on May 21, 2004.

(51) Int. Cl. A61K 31/513; A61K 31/42

(52) U.S. Cl. 514/269

(57) ABSTRACT

Provided herein are compositions comprising a pyrimidine synthesis inhibitor and a pharmaceutically acceptable carrier Such compositions can be used in methods of increasing Na⁺ dependent fluid clearance by a pulmonary epithelial cell; of treating a pulmonary disease in a subject; of reducing one or more symptoms or physical signs of a respiratory syncytial virus infection in a subject; of identifying a subject at risk for respiratory syncytial virus infection and administering to the subject a composition comprising an effective amount of a pyrimidine synthesis inhibitor; of identifying a subject with a respiratory syncytial virus infection and administering to the subject a composition comprising a pyrimidine synthesis inhibitor in an amount effective to reduce Na⁺ dependent alveolar fluid in the subject; and of screening for a test compound that increases Na⁺ dependent fluid uptake by a pulmonary epithelial cell.

### CROSS-REFERENCE TO RELATED APPLICATION

This application claims the benefit of U.S. Provisional Application No. 60/573,558 filed on May 21, 2004, which is hereby incorporated herein by reference in its entirety.

### ACKNOWLEDGEMENTS

This invention was made with government support under Grants Nos. RR17626, HL31197, HL075540, HL51173 and HL72817 from the National Institutes of Health. The government has certain rights in the invention.

### BACKGROUND

Respiratory syncytial virus (RSV) is the most common cause of lower respiratory tract (LRT) disease in infants and children worldwide, and may also be under-diagnosed as a cause of community-acquired LRT infections among adults.

During the years 1980-1996, an estimated 1.65 million hospitalizations for bronchiolitis occurred among children younger than 5 years, accounting for 7.0 million inpatient days. Fifty-seven percent of these hospitalizations occurred among children younger than 6 months and 81% among those younger than 1 year. Among children younger than 1 year, annual bronchiolitis hospitalization rates increased 2.4-fold, from 12.9 per 1000 in 1980 to 31.2 per 1000 in 1996. The proportion of hospitalizations for lower respiratory tract illnesses among children younger than 1 year associated with bronchiolitis increased from 22.2% in 1980 to 47.4% in 1996; among total hospitalizations, this proportion increased from 5.4% to 16.4%. An estimated 51,240 to 81,985 annual bronchiolitis hospitalizations among children younger than 1 year were related to RSV infection. If hospitalizations for bronchiolitis with pneumonia are also considered, RSV infection accounts for up to 126,000 hospitalizations per year in the United States alone. Currently, there is no effective treatment for RSV.

Rhinorrhea, pulmonary congestion and hypoxemia are significant components of most respiratory infections, including RSV infection, but the mechanisms underlying altered lung fluid dynamics in such diseases are poorly understood. Moreover, epidemiologic studies suggest a strong link between severe respiratory syncytial virus (RSV)-induced bronchiolitis in infancy and allergic disease. RSV infection is also of major importance in cattle where such infections can result is severe respiratory tract disease.

Needed in the art are improved methods and compositions for preventing and treating respiratory infections including RSV infections.

### SUMMARY OF THE INVENTION

Provided herein are compositions comprising a pyrimidine synthesis inhibitor and a pharmaceutically acceptable canier. The compositions are suitable for topical administration to a pulmonary epithelial cell of a subject. Also provided herein is a device comprising at least one metered dose of a composition comprising a therapeutic amount of a pyrimidine synthesis inhibitor. Each metered dose comprises a therapeutic amount or a portion thereof of the pyrimidine synthesis inhibitor for treating a pulmonary disease in a subject.

Further provided herein are methods of increasing Na⁺ dependent fluid clearance by a pulmonary epithelial cell, of treating a pulmonary disease in a subject, of reducing one or more symptoms or physical signs of a respiratory syncytial virus infection in a subject, of identifying a subject at risk for respiratory syncytial virus infection and administering to the subject a composition comprising an effective amount of a pyrimidine synthesis inhibitor, of identifying a subject with a respiratory syncytial virus infection and administering to the subject a composition comprising a pyrimidine synthesis inhibitor in an amount effective to reduce Na⁺ dependent alveolar fluid in the subject, and of screening for a test compound that increases Na⁺ dependent fluid uptake by a pulmonary epithelial cell.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BREIF DESCRIPTION OF THE DRAWINGS

The accompanying drawings, which are incorporated in and constitute part of the specification, illustrate several aspects described below.

FIG. 1 is a schematic diagram illustrating the pyrimidine and purine biosynthesis pathways.

FIG. 2 shows the effect of RSV infection on peripheral oxygenation. (A) Timecourse of effect of RSV infection on SmO₂ (mixed oxygen saturation) in conscious BALB/c mice (n=10-36 per day). (B) Sample 3-lead ECG (electrocardiogram) tracings at beginning and end of alveolar fluid clearance (AFC) period for mock-infected mouse and RSV-infected mouse at d2. (C) Effect of RSV infection on % ΔHR30 at d2 (n=17 for mock-infected mice; n=11 for RSV-infected mice). *p<0.05, compared with mock-infected mice.

FIG. 3 shows the effect of RSV infection on nasal potential difference (NPD) in BALB/c mice. (A) Representative tracings of NPD in a mock-infected mouse and an RSV-infected mouse at d4. (B) Effect of RSV infection on basal NPD. (C) Effect of RSV infection on the amiloride-sensitive component of NPD (NPDAMIL). (D) Sample tracing of change in NPD with application of ±60 nA pulses to nasal epithelium in a mock-infected mouse and an RSV-infected mouse at d4. (E) Effect of RSV infection on ΔNPD following application of ±60 nA pulses to nasal epithelium. n=5-9 for all groups. Dashed line on sample tracings indicates 0 mV on the chart, arrow indicates time of 100 μM amiloride addition. *p<0.05, **p<0.005, compared with mock-infected animals.

FIG. 4 shows the effect of nucleotide synthesis inhibition on body weight after RSV infection. (A) Effect of leflunomide (an inhibitor of UTP synthesis) on acute weight loss after RSV infection in BALB/c mice (n=35 for untreated mice; n=19 for leflunomide-treated mice). (B) Effect of 6-MP treatment on acute weight loss after RSV infection in BALB/c mice (n=35 for untreated mice; n=30 for 6-MP-treated mice). *p<0.05, **p<0.005, ***p<0.0005, compared with body weight in untreated mice at each timepoint.

FIG. 5 shows the effect of gavage of mice with leflunomide (LEF) reverses RSV-mediated inhibition of AFC at day 2 p.i. The effect of LEF is prevented by concomitant administration of uridine.

FIG. 6 shows the effect of gavage of mice with leflunomide (LEF) reverses RSV-induced increased in lung water content at day 2 p.i. The effect of leflunomide is prevented by concomitant administration of uridine. Importantly, treatment of mice with LEF and/or uridine has no effect on virus replication in lung tissue at day 2 p.i.

FIG. 7 shows the effects of addition of a wide spectrum of inhibitors of volume-regulated anion channels (VRACs) to the AFC instillate reverses RSV mediated inhibition of AFC at day 2 p.i.

FIG. 8 shows the effect of nucleotide synthesis inhibition on lung water content after RSV infection. (A) Effects of leflunomide and uridine treatment on lung water content at d2 (n=7-8 for all groups). (B) Effect of 6-MP on lung water content at d2 (n=8 for uninfected mice; n=7 for untreated, RSV-infected mice; n=15 for 6-MP-treated mice). Lung water content was measured by wet:dry weight ratio. ***p<0.0005, compared with wet:dry weight ratio in uninfected mice.

FIG. 9 shows the effect of nucleotide synthesis inhibition on RSV replication in mouse lungs. (A) Effects of leflunomide and ulidine treatment on virus replication at d2 (n=6 for untreated, uridine-treated and leflunomide-and uridine-treated mice; n=12 for leflunomide-treated mice). (B) Effect of 6-MP on virus replication at d2 (n=6 for untreated mice; n=12 for 6-MP-treated mice). (C) Effects of continued leflunomide treatment on virus replication at d8 (n=6 for untreated mice; n=12 for leflunomide-treated mice). (D) Effects of leflunomide treatment to d2 on virus replication at d8 (n=6 for both groups). Dashed line indicates limits of detection of assay. ***p<0.0005, compared with viral titer in untreated mice.

FIG. 10 shows the effect of leflunomide treatment on hypoxemia after RSV infection. (A) Effect of leflunomide treatment on SmO₂ in mice at d2 (n=8 for untreated, RSV-infected mice; n=7 for leflunomide-treated, RSV-infected mice). (B) Effect of RSV infection and leflunomide treatment on % ΔHR30 at d2 (n=11 for untreated, RSV-infected mice; n=9 for leflunomide-treated, RSV-infected mice). *p<0.05, compared with untreated values.

FIG. 11 shows the effects of leflunomide treatment on NPD in BALB/c mice. (A) Sample tracing of NPD in a leflunomide-treated, RSV-infected mouse at d4. (B) Effect of leflunomide treatment on basal NPD in RSV-infected mice. (C) Effect of leflunomide treatment on NPDAMIL in RSV-infected mice. n=5-9 for all groups. Dashed line on sample tracings indicates 0 mV on the chart, arrow indicates time of 100 μM amiloride addition. *p<0.05, **p<0.005, compared with NPD in untreated animals.

FIG. 12 shows that infection with RSV significantly inhibits basal alveolar fluid clearance (AFC) at days 2 and 4 post infection (p.i.). Mock infection (M) has no effect on AFC, compared to uninfected mice (U). Basal AFC was inhibited by 43% (from mock-infected values) at day 2 and by 26% at day 4. Amiloride sensitivity of AFC was also reduced at day 1, and absent at days 2 and 4 p.i..

FIG. 13 shows that the addition of an inhibitor of dihydro-orotate reductase (25 μm A77-1726) to the AFC instillate reverses RSV-mediated inhibition of AFC at day 2 p.i. The effect of A77-1726 is fully reversed by concomitant addition of 50 mM uridine to the AFC instillate, but is not recapitulated by 25 mM genistein (a nonspecific tyrosine kinase inhibitor).

FIG. 14 shows that addition of inhibitors of IMP dehydrogenase (25 μm 6-MP or MPA) to the AFC instillate has only a minor effect on RSV-mediated inhibition of AFC at day 2 p.i. The small effect of IMP dehydrogenase inhibitors is a consequence of depletion of ATP, which is a necessary precursor for de novo pyrimidine synthesis. The MPA effect was fully reversed by concomitant addition of 50 mM hypoxanthine (HXA) to the AFC instillate, allowing synthesis of ATP via the purine salvage pathway.

### DETAILED DESCRIPTION OF THE INVENTION

The present invention may be understood more readily by reference to the following detailed description of preferred embodiments of the invention and the Examples included therein and to the FIGS. and their previous and following description.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used in the specification and the appended claims, the singular forms “a,” “an” and “the” include plural referents unless the context clearly dictates otherwise. Thus, for example, reference to “a pyrimidine synthesis inhibitor” includes mixtures of one or more pyrimidine synthesis inhibitors, and the like. Similarly, reference to “a pulmonary epithelial cell” includes one or more pulmonary epithelial cells. Thus, for example, a composition suitable for administration to “a pulmonary epithelial cell” is suitable for administration to one or more such cells.

Abbreviations may be used throughout and have the following meanings. Such abbreviations include, but are not limited to AFC (Alveolar fluid clearance), ALF (Airspace lining fluid), BALF (Bronchoalveolar lavage fluid), ΔNPD (change in nasal potential difference), DHOD (Dihydro-orotate dehydrogenase), HXA (Hypoxanthine), HRSTART (Heart rate at start of ventilation period), HREND (Heart rate at end of ventilation period), LEF (Leflunomide), MPA (mycophenolic acid), 6-MP (6-mercaptopurine), NPD (Nasal potential difference), NPDAMIL (Amiloride-sensitive component of nasal potential difference), NRte (Nasal transepithelial resistance), % ΔHR30 (% change in rate over 30-minute ventilation period), P2YR (P2Y purinergic nucleotide receptor), RSV (Respiratory syncytial virus), SmO₂ (Mean hemoglobin O₂ saturation), and VRAC (Volume-regulated anion channel). Moreover, other abbreviations may also be used, which would be clear to one skilled in the art and/or are clear from the context in which the given abbreviation is used.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a bovine species such as, for example, Bos taurus, Bos indicus, or crosses thereof. In another aspect, the subject is a mammal such as a primate or a human.

“Optional” or “optionally” means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

The terms “higher,” “increases,” “elevates,” or “elevation” refer to increases above a control value (e.g., a basal level). The terms “low,” “lower,” “reduces,” or “reduction” refer to decreases below a control value (e.g., a basal level). For example, basal levels are normal in vivo levels prior to, or in the absence of, addition of an agent such as, leflunomide, A77-1726 or another pyiimidine synthesis inhibitor. Control levels can also include levels from a subject or sample in the absence of a disease state. The control value can be determined from the same subject(s) or sample(s) prior to or after disease or treatment. The control value can be from a different subject(s) or sample(s) in the absence of the disease or treatment.

Provided herein are compositions comprising a pyrimidine synthesis inhibitor and a pharmaceutically acceptable carrier. Such compositions can be used in methods of increasing Na⁺ dependent fluid clearance by a pulmonary epithelial cell; of treating a pulmonary disease in a subject; of reducing one or more symptoms or physical signs of a respiratory syncytial virus infection in a subject; of identifying a subject at risk for respiratory syncytial virus infection and administering to the subject a composition comprising an effective amount of a pyrimidine synthesis inhibitor; of identifying a subject with a respiratory syncytial virus infection and administering to the subject a composition comprising a pyrimidine synthesis inhibitor in an amount effective to reduce Na⁺ dependent alveolar fluid in the subject; and of screening for a test compound that increases Na⁺ dependent fluid uptake by a pulmonary epithelial cell, which are described in greater detail below.

As used herein “treating” includes the reduction of symptoms or physical signs of a given respiratory infection in the subject. Thus, the disclosed compositions and methods can be used to reduce one or more symptoms or physical signs of a respiratory infection in a subject. Such symptoms and physical signs include, but are not limited to, rhinorrhea, hypoxemia, pulmonary edema, decreased cardiac function, cough, weight loss, wheezing, cachexia, and pulmonary congestion.

One exemplary disease, for which treatment with the disclosed compositions and methods is useful, is respiratory syncytial virus (RSV) infection. RSV inhibits Na⁺-dependent alveolar fluid clearance (AFC) in BALB/c mice, and both P2Y nucleotide receptor antagonists and pyrimidinolytic enzymes prevent inhibition of AFC. RSV infection results in release of both UTP and ATP into the ALF and the reduction in AFC is associated with the early phase of RSV infection resulting in significant physiologic impairment of the host.

In the lungs of infants ventilated for severe RSV infection, levels of surfactant proteins SP-A and SP-D are reduced, the amount of the major surfactant phospholipid, dipalmitylphosphatidylcholine, is lowered, and the biophysical surface activity of the surfactant recovered is impaired in comparison to control infants (Kerr and Patton, 1999). Although beta adrenergic receptor agonists (βARA) bronchodilator agents have been used improve alveolar fluid clearance in adult respiratory distress syndrome by elevating intracellular cAMP, beta adrenergic receptor-mediated signaling in the respiratory epithelium is abnormal following RSV infection, which may account for the poor efficacy of βARA in RSV therapy Thus, in the case of RSV infection, the disclosed methods and compositions are used to increase levels of surfactant phospholipids, like dipalhnitylphosphatidylcholine, in infants or to improve the efficacy of beta adrenergic receptor agonists (βARA) bronchodilator agents.

Furthermore, epidemiologic studies suggest a strong link between severe respiratory syncytial virus (RSV)-induced bronchiolitis in infancy and allergic disease. The compositions and methods provided herein are useful during RSV infection to reduce RSV-induced airway hypersensitivity and predisposition to subsequent development of asthma. RSV infection is also of major importance in cattle (i.e. in Bos taurus and Bos indicus, or in crosses thereof) and can result is severe respiratory tract disease.

Alveolar fluid clearance is related to ion transport in pulmonary epithelial cells. For example, the alveolar epithelial wall consists of two types of cells: type I cells and type II cells. Type I cells cover the largest fraction of the alveolar epithelium (about 95%). Type II cells produce surfactant. It is currently believed that both type I and type II cells transport sodium ions in an active manner. The sodium-potassium pump, located in the basolateral surface of the epithelial cells, sets up an electrochemical gradient across the apical membrane which favors sodium ions to enter from the alveolar space into the cytoplasm. Sodium crosses the alveolar epithelium mainly through proteins called channels. Once in the cytoplasm they are extruded across the basolateral membrane by the sodium-potassium pump which utilizes ATP. To preserve electrical neutrality, chloride ions follow the movement of sodium ions through pathways located either between cells or through cellular channels. The movement of ions creates an osmotic pressure difference between the interstitial and alveolar space favoring the reabsorption of fluid.

Active sodium transport plays an important role in limiting the amount of fluid in the alveolar space in a number of pathological conditions (viral infections, pneumonias, acute lung injury etc). Under basal conditions, the dominant ion transport process of respiratory epithelia is active, amiloride-sensitive, transport of Na⁺ ions from the lumenal fluid to the interstitial space underlying the epithelium. Na⁺ ions in the alveolar lining fluid (ALF) passively diffuse into bronchoalveolar epithelial cells predominantly through the cation and Na⁺-selective, amiloride-sensitive epithelial Na⁺ channel (ENaC) in the apical membrane. Cl⁻ ions follow Na⁺ movement via paracellular pathways, or possibly via the cystic fibrosis transmembrane regulator (CFTR), to maintain electrical neutrality. Transport of NaCl creates a transepithelial osmotic gradient. Since the transepithelial water permeability of the respiratory epithelium is high, the gradient causes water to move passively from the airspace to the interstitium, thereby clearing airspace fluid.

RSV-mediated inhibition of AFC is associated with hypoxemia, impaired cardiac function and increased UTP and ATP content of bronchoalveolar lavage fluid. Moreover, despite the absence of a direct antiviral effect on RSV replication in the lungs, systemic inhibition of de novo pyrimidine synthesis with leflunomide improves not only AFC and lung water content, but also physiologic impairments (including, reduced body weight, depressed SmO₂ and cardiac function, and altered nasal potential difference) in RSV-infected mice. RSV-mediated inhibition of AFC can be prevented by pharmacologic blockade of volume regulated anion channels (VRACs) showing that de novo UTP synthesis and release through VRACs is necessary for RSV-mediated inhibition of AFC to occur, and demonstrating that the de novo pyrimidine synthesis and release pathway is an attractive target for inhibitor therapies designed to alleviate the symptoms of RSV infection or other respiratory infections.

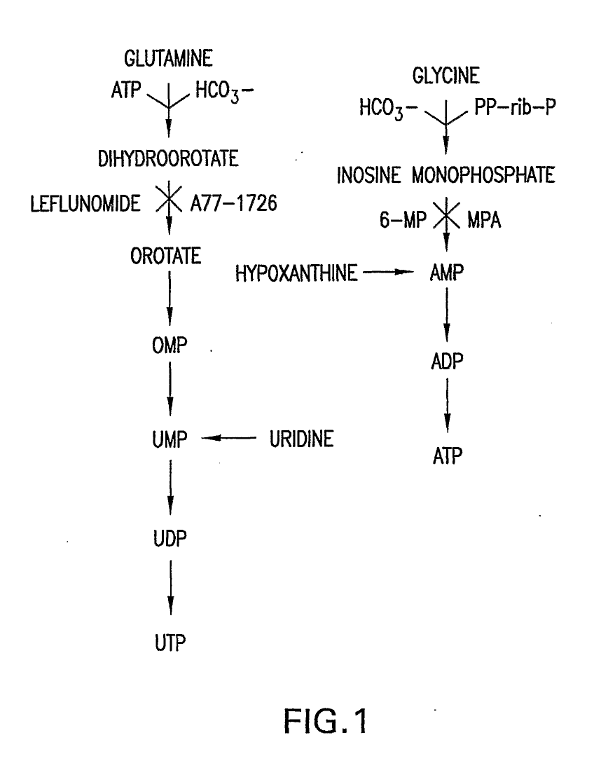

FIG. 1 is a schematic diagram illustrating the pyrimidine and purine biosynthesis pathways. UTP is synthesized de novo from glutamine, ATP and HCO³⁻. UTP can also be synthesized fiom uridine via a salvage pathway. Leflunomide and its active metabolite, A77-1726, both inhibit activity of dihydro-orotate dehydrogenase, which converts dihydro-orotate to orotate. Both agents block de novo pyrimidine synthesis but have no effect on the pyrimidine salvage pathway, or on purine synthesis.

Optionally, the composition comprises a pyrimidine syntlhesis inhibitor that is leflunomide. Optionally, the composition comprises a pyrimidine synthesis inhibitor that is A77-1726. Optionally, the composition comprises a combination of leflunomide and A77-1726 and/or a combination of leflunomide or A77-1726 with another pyrimidine synthesis inhibitor. Leflunomide, a prodrug whose active metabolite is A77-1726, is used for treatment of rheumatoid arthritis, under the trade name ARAVA® (Aventis Pharmaceuticals, Bridgewater, N.J.). Both leflunomide and A77-1726 act as inhibitors of the enzyme dihydro-orotate reductase (also known as dihydro-orotate dehydrogenase or dihydro-orotase), which is a component of the trifunctional enzyme complex CAD (carbamyl phosphate synthetase, aspartate transcarbamylase, and dihydro-orotase), a central component of the de novo pyrimidine synthesis pathway. Thus, as with leflunomide and A77-1726, the composition can be an inhibitor of dihydro-orate reductase.

The compositions can be administered in vivo in a pharmaceutically acceptable carrier. By “pharmaceutically acceptable” is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials may be in solution, suspension (for example, incorporated into microparticles, liposomes, or cells). These may be targeted to a particular cell type via antibodies, receptors, or receptor ligands.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carriers include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered. For example, it is within the skill in the art to choose a particular carrier suitable for inhalational and/or intranasal administration, or for compositions suitable for topical administration to a pulmonary epithelial cell.

The compositions may also include thickeners, diluents, buffers, preservatives, surface active agents and the like in addition to the compositions and carriers. The compositions may also include one or more active ingredients such as antimicrobial agents, anti-inflammatory agents, anesthetics, and the like.

The disclosed compositions are suitable for topical administration to a pulmonary epithelial cell or to a plurality of pulmonary epithelial cells of a subject. Thus, the compositions comprising a pyrimidine synthesis inhibitor are optionally suitable for administration via inhalation, (i.e., the composition is an inhalant). Further, the compositions are optionally aerosolized. And, further still, the compositions are optionally nebulized. Administration of the compositions by inhalation can be through the nose or mouth via delivery by a spraying or droplet mechanism. Delivery can also be directly to any area of the respiratory system (e.g., lungs) via intubation. Optionally, the pulmonary epithelial cell to which a composition is administered is located in the nasal cavity, nasal passage, nasopharynx, pharynx, trachea, bronchi, bronchiole, or alveoli of the subject. Optionally, the pulmonary epithelial cell to which a composition is administered is a bronchoalveolar epithelial cell. Moreover, if the compositions are administered to a plurality of pulmonary epithelial cells, the cells may be optionally located in any or all of the above anatomic locations, or in a combination of such locations.

Topical administration to a pulmonary epithelial cell accordingly may be made by pulmonary delivery through nebulization, aerosolization or direct lung instillation. Thus, compositions suitable for topical administration to a pulmonary epithelial cell in a subject include compositions suitable for inhalant administration, for example as a nebulized or aerosolized preparation. For example, the compositions may be administered to an individual by way of an inhaler, e g., metered dose inhaler or a dry powder inhaler, an insufflator, a nebulizer or any other conventionally known method of administering inhalable medicaments.

The compositions of the present invention may be an inhalable solution. The inhalable solution may be suitable for administration via nebulization. The compositions may also be provided as an aqueous suspension. Optionally, the formulation of the present invention comprises a therapeutically effective amount of a pyrimidine synthesis inhibitor in an aqueous suspension.

Optionally, the compositions may be administered by way of a pressurized aerosol comprising, separately, a pyrimidine synthesis inhibitor, or salt or an ester thereof with at least a suitable propellant or with a surfactant or a mixture of surfactants. Any conventionally known propellant may be used.

Also provided herein are combinations comprising a composition provided herein and a nebulizer. Also disclosed herein are containers comprising the agents and compositions taught herein. The container can be, for example, a nasal sprayer, a nebulizer, an inhaler, a bottle, or any other means of containing the composition in a form for administration to a mucosal surface. Optionally, the container can deliver a metered dose of the composition.

Any nebulizer can be used with the disclosed compositions and methods. In particular, the nebulizers for use herein nebulize liquid formulations, including the compositions provided herein, containing no propellant. The nebulizer may produce the nebulized mist by any method known to those of skill in the art, including, but not limited to, compressed air, ultrasonic waves, or vibration. The nebulizer may further have an internal baffle. The internal baffle, together with the housing of the nebulizer, selectively removes large droplets from the mist by impaction and allows the droplets to return to the reservoir. The fine aerosol droplets thus produced are entrained into the lung by the inhaling air/oxygen.

Thus, nebulizers that nebulize liquid formulations containing no propellant are suitable for use with the compositions provided herein. Examples of such nebulizers are known in the art and are commercially available. Nebulizers for use herein also include, but are not limited to, jet nebulizers, ultrasonic nebulizers, and others. Exemplary jet nebulizers are known in the art and are commercially available.

The compositions may be sterile filtered and filled in vials, including unit dose vials providing sterile unit dose formulations which are used in a nebulizer and suitably nebulized. Each unit dose vial may be sterile and suitably nebulized without contaminating other vials or the next dose.

Optionally, the disclosed compositions are in a form suitable for intranasal administration. Such compositions are suitable for delivery into the nose and nasal passages through one or both of the nares and can comprise delivery by a spraying mechanism or droplet mechanism, or through aerosolization.

If the compositions are used in a method wherein topical pulmonary administration is not used, the compositions may be administered by other means known in the art for example, orally, parenterally (e.g., intravenously), by intramuscular injection, by intraperitoneal injection, and transdermally.

Further provided herein is a device comprising at least one metered dose of a composition comprising a therapeutic amount of a pyrimidine synthesis inhibitor wherein each metered dose comprises a therapeutic amount or a portion thereof of the pyrimidine synthesis inhibitor for treating a pulmonary disease in a subject. The pyrimidine synthesis inhibitor can comprise a pyrimidine synthesis inhibitor as disclosed above, or combinations thereof.

Also provided herein is a method of increasing Na⁺ dependent fluid clearance by a pulmonary epithelial cell comprising contacting the cell with an effective amount of a pyrimidine synthesis inhibitor. The contacting causes increased Na⁺ dependent fluid clearance by the cell. Optionally, the pulmonary epithelial cell is contacted in vivo. Optionally, the pulmonary epithelial cell is contacted in vitro.

Further provided is a method of treating a pulmonary disease in a subject comprising, contacting a plurality of pulmonary epithelial cells in the subject with an effective amount of a pyrimidine synthesis inhibitor. The effective amount of the pyrimidine synthesis inhibitor causes increased Na⁺ dependent alveolar fluid clearance in the subject. The method can be used wherein the subject has or is at risk of developing respiratory syncytial virus infection. Other pulmonary pathogens that cause disease for which the disclosed method can be used include but are not limited to Paramyxoviruses (Respiratory syncytial virus [human and bovine], metapneumovirus, parainfluenza, measles), Orthomyxoviruses (Influenza A, B, and C viruses), Poxviruses (Smallpox, monkeypox), New world hantaviruses, Rhinoviruses, Coronaviruses (Severe acute respiratory syndrome agent), Herpesviruses (Herpes simplex virus, cytomegalovirus), Streptococcus pneumoniae, Hemophilus influenzae, Pseudomonas aeruginosa, Mycobacterium tuberculosis, Mycoplasma pneumoniae, Bacillus anthracis, Legionella pneumophila, Klebsiella pneumoniae, Chlamydia, Listeria monocytogenes, Pasteurella multocida, and Burkholderia cepacia.

Further provided is a method of reducing one or more symptoms or physical signs of a respiratory syncytial virus infection in a subject at risk for a respiratory syncytial virus infection comprising, administering to the subject a composition comprising an effective amount of a pyrimidine synthesis inhibitor. As described above, such symptoms or physical signs, include, but are not limited to rhinorrhea, hypoxemia, pulmonary edema, decreased cardiac function, cough, weight loss, wheezing, cachexia, and pulmonary congestion. A subject at risk for a respiratory syncytial virus infection can be readily determined by one skilled in the art. For example, such a determination could be made by a physician or veterinarian based on a subject's medical history, presenting symptoms/physical signs, physical exam, diagnostic tests or any combination thereof.

Also provided is a method comprising, identifying a subject at risk for respiratory syncytial virus infection and administering to the subject a composition comprising an effective amount of a pyrimidine synthesis inhibitor. Moreover, provided is a method comprising, identifying a subject with a respiratory syncytial virus infection and administering to the subject a composition comprising a pyrimidine synthesis inhibitor in an amount effective to reduce Na⁺ dependent alveolar fluid in the subject.

In the disclosed methods, the pyrimidine synthesis inhibitor is optionally leflunomide, A77-1726, or combinations thereof. Further, leflunomide and/or A77-1726 can be used in the disclosed methods in combination with one or more other pyrimidine synthesis inhibitors.

The terms “effective amount” and “effective dosage” or “therapeutic amount” are used interchangeably. The term “effective amount” is defined as any amount necessary to produce a desired physiologic response. Effective amounts and schedules for administering the compositions used in the disclosed methods may be determined empirically, and making such determinations is within the skill in the art. The effective dosage ranges for the administration of the compositions used in the disclosed methods are those large enough to produce the desired effect in which the symptoms of the disorder are affected. The dosage should not be so large as to cause adverse side effects, such as unwanted cross-reactions, anaphylactic reactions, and the like.

Generally, the therapeutic amount or dosage will vary with the age, condition, sex and extent of the disease in the subject, route of administration, or whether other drugs are included in the regimen, and can be determined by one of skill in the art. The dosage can be adjusted by the individual physician or veterinarian in the event of any counterindications. Dosage can vary, and can be administered in one or more dose administrations daily, for one or several days. The effective amount of the compositions used in the disclosed methods required may vary depending on the method used and on the airway disorder being treated, the particular pyrimidine synthesis inhibitor and or carrier used, and mode of administration, and the like. Thus, it is not possible to specify an exact amount for every composition. However, an appropriate amount can be determined by one of ordinary skill in the art using only routine experimentation given the teachings herein. For example, the dihydro-oroate reductase or pyrimidine synthesis inhibitor used in vivo can be administered at a dose of about 10-50 mg/kg, at a dose of about 25-45 mg/kg, or at a dose of about 30-40 mg/kg.

A77-1726, leflunomide, and/or other pyrimidine inhibitor therapeutic amounts, effective amounts, or effective dosages can be administered by aerosol at reasonable intervals and remain effective. For example, an effective dose of the compositions described herein can be administered S.I.D., B.I.D., Q.I.D., or once or more an hour for a day, several days, a week or more. Thus, for example, the compositions can be administered once every 1, 2, 4, 8, 12, or 24 hours, or combinations or intervals thereof, for a duration of 1 day, 2 days, 3 days, 4 days, 5 days, 6 days, or for 1 week or more or any interval or combination thereof. By interval is meant any increment of time within the provided values. Thus, the composition, for example, can be administered every three hours over 12 hours and so forth. Optionally, the composition is administered once. Such time courses could be determined by one of skill in the art using, for example, the parameters described above for determining an effective dose.

The efficacy of administration of a particular dose of the compositions according to the methods described herein can be determined by evaluating the particular aspects of the medical history signs, symptoms, and objective laboratory tests that are known to be useful in evaluating the status of a subject with pulmonary infection, such as a RSV infection, or one that is at risk of contracting such an infection. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: 1) a subject's physical condition is shown to be improved (e.g., pulmonary congestion is reduced or eliminated), 2) the progression of the disease, infection, is shown to be stabilized, slowed, or reversed, or 3) the need for other medications for treating the disease or condition is lessened or obviated, then a particular treatment regimen will be considered efficacious. Such effects could be determined in a single subject in a population (e.g., using epidemiological studies).

The teachings herein can also be used in methods of screening. For example, provided herein is a method of screening for a test compound that increases Na⁺ dependent fluid uptake by a pulmonary epithelial cell comprising contacting a pulmonary epithelial cell with the test compound in the presence of an excess of UTP, detecting Na⁺ dependent fluid uptake by the pulmonary epithelial cell, an increase in Na⁺ dependent fluid uptake as compared to a control indicating a test compound that increases Na⁺ dependent fluid uptake by a pulmonary epithelial cell. Optionally, the cells are contacted in vivo. Optionally, the cells are contacted in vitro. The method may optionally further comprise removing the UTP and detecting reversibility of the increase in Na⁺ dependent fluid uptake.

In another screening method example, a method of screening for a test compound that increases Na⁺ dependent fluid uptake comprises contacting the test compound with a cell that expresses a heterologous nucleic acid that encodes a pyrimidine synthesis gene, and detecting Na⁺ dependent fluid uptake by the cell, an increase in Na⁺ dependent fluid uptake as compared to a control level, indicating a test compound that increases Na⁺ dependent fluid uptake. Optionally, the cells are contacted in vivo. Optionally, the cells are contacted in vitro.

Another method of screening for a test compound that increases Na⁺ dependent fluid uptake by a respiratory epithelial cell comprises infecting a H441 cell or cell line with RSV, contacting the infected cell or cell line with the test compound, and measuring ion transport across the infected cell or cells of the infected cell line. An increase in ion transport across an infected H441 cell or cell line when compared to a control indicates that a test compound that increased Na⁺ dependent fluid uptake. Ion transport can be compared to ion transport across a control cell or cell line, which optionally may be a non-RSV infected H441 cell line, or may be an infected H441 cell or cell line in the absence of the test compound. Optionally, the test compound comprises a pyrimidine synthesis inhibitor. Optionally, the cells are contacted in vivo. Optionally, the cells are contacted in vitro.

### EXAMPLES

The following examples are put forth so as to provide those of ordinary skill in the art with a complete disclosure and description of the methods claimed herein, and are intended to be purely exemplary of the invention and are not intended to limit the scope of what the inventors regard as their invention except as and to the extent that they are included in the accompanying claims. Efforts have been made to ensure accuracy with respect to numbers (e.g., amounts, temperature, etc.), but some errors and deviations should be accounted for.

Methods

Preparation of viral inocula and infection of mice. Preparation of viral stocks and intranasal infection of eight to twelve week-old pathogen-free BALB/c mice of either sex with RSV strain A2 (106 PFU in 100 μl) were performed as described in Davis et al., “Nucleotide-mediated inhibition of alveolar fluid clearance in BALB/c mice after respiratory syncytial virus infection,” Am. J. Physiol. Lung Cell Mol. Physiol. 286:L112-L120. (2004). Data for each experimental group was derived from a minimum of 2 independent infections.

Measurement of mean peripheral blood oxygen saturation. Peripheral blood oxygen saturation was measured in conscious mice, using a PREEMIE OXYTIP® sensor (Datex-Olmeda, hic., Madison, Wis.), connected to a TUFFSAT™ pulse oximeter (Datex-Ohmeda, Inc., Madison, Wis.). Because of the extremely rapid pulse rate of the mouse, oximetry values are mean hemoglobin O₂ saturation (SmO₂) values from arterial and venous blood.

Measurement of heart rate changes with ventilation. ECG tracings were used to measure heart rate (number of QRS complexes/cm) at the start (HRSTART) and end (HREND) of the AFC assay. The % change in rate over the 30-minute ventilation period (% ΔHR30) was calculated as (HRSTART−HREND)/HRSTART.

Measurement of nasal potential difference. The potential difference across the nares of anesthetized mice (with the tail as reference) was measured as previously described Grubb et al., (1994) “Hyperabsorption of Na+ and raised Ca(2+)-mediated Cl-secretion in nasal epithelia of CF mice,” Am. J. Physiol 266:C1478-C1483. A baseline NPD was recorded during perfusion of the nasal epithelium with lactated Ringer's solution. The amiloride-sensitive component of NPD (NPDAMIL) was determined by perfusion with lactated Ringer's solution containing 100 μM amiloride. Current pulses of ±60 nA were applied across the epithelium by a 12V battery in series with a 200 MΩ resistor. Changes in NPD in response to the current pulses (proportional to nasal transepithelial resistance, NRte) were recorded (ΔNPD).

Bronchoalveolar lavage. Bronchoalveolar lavage fluid (BALF) was collected as previously described in Davis et al., (2004), using 1 ml of sterile normal saline for cytokine ELISAs, or 0.3 ml of sterile saline for nucleotide assays. Lavagates were centrifuged to remove cells and supernatants stored at −80° C.

Measurement of nucleotides in BALF. Endogenous nucleotidases in BALF were heat denatured (100° C., 3 minutes) and UTP/ATP content measured using the UDP-glucose pyrophosphorylase and luciferine-luciferase assays, respectively.

Measurement of heme in BALF. BALF heme content was measured spectrophotemetrically using the Drabkins assay.

Systemic inhibition of de novo pyrimidine and purine synthesis. Leflunomide (5-methylisoxazole-4-[4-trifluoromethyl]carboxanilide, 35 mg/kg in distilled water containing 1% methylcellulose) was administered once daily by oral gavage in a volume of 300 μl/mouse for 8 days prior to infection, then throughout the infection period. Vehicle controls were gavaged with an equivalent volume of 1% methylcellulose in distilled water. Uridine (1 g/kg in 0.9% NaCl) was administered by i.p. injection every 12 hours in a volume of 100 μl (8). 6-MP (35 mg/kg in 1N NaOH, pH adjusted to 7.9 with 2 M Na2HPO4) was administered by i.p. injection every 24 hours in a volume of 100 μl, for 5 days prior to infection, then throughout the infection period.

Measurement of proinflammatory cytokines in BALF. Cytokine levels were determined using Quantikine M ELISA kits (R & D Systems), in accordance with manufacturer's instructions.

Statistical Analyses. Descriptive statistics were calculated using Instat software (GraphPad, San Diego, Calif.). Differences between group means were analyzed by ANOVA or Student's t test, with appropriate post tests. All data values are presented as mean ±SE.

Results

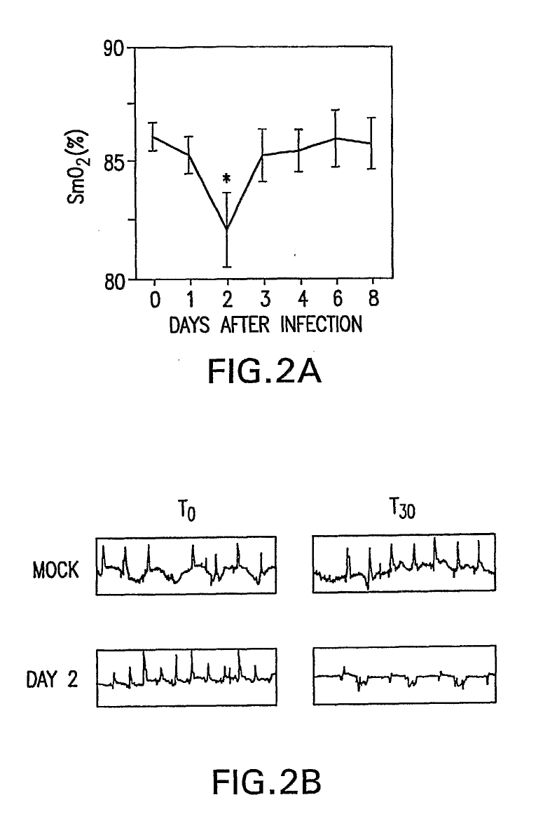

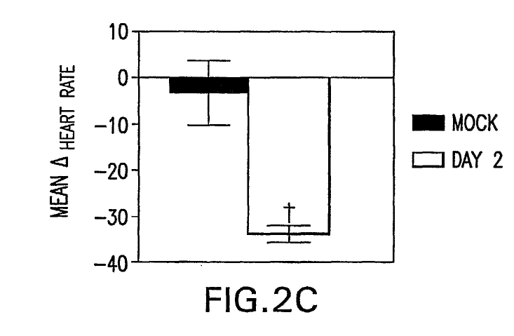

Effect of RSV infection on peripheral blood oxygenation. Impairment of basal AFC at d2 was associated with a small but significant reduction in peripheral blood SmO₂ compared to mock-infected animals (FIG. 2A). No decline in SmO₂ was found at other timepoints.

As an additional index of hypoxemia, 3-lead ECG recordings were evaluated from mock-infected and RSV-infected mice at d2 for evidence of alterations in heart rate during the course of AFC measurement (% ΔHR30). Infection with RSV was associated with a significant increase in % ΔHR30 at d2 (FIGS. 2B and 2C). There was no difference in duration of anesthesia between the two groups.

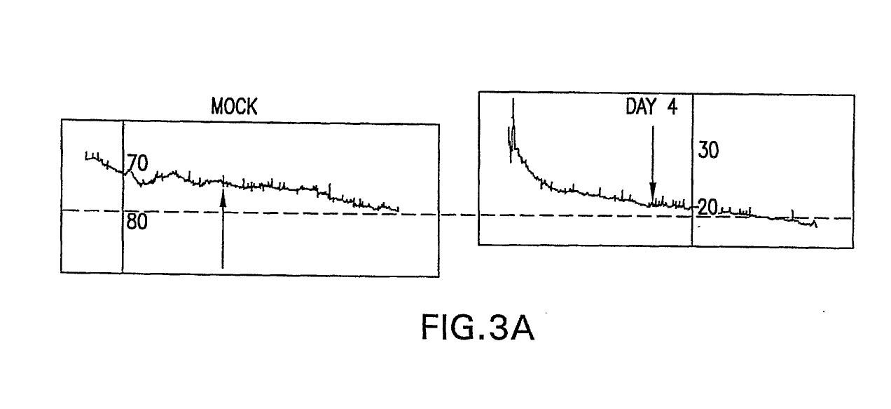

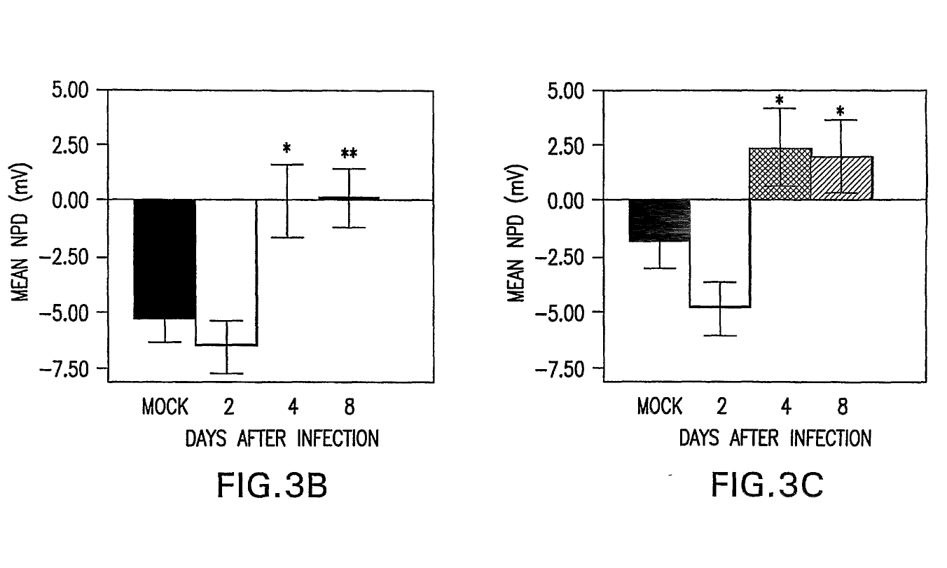

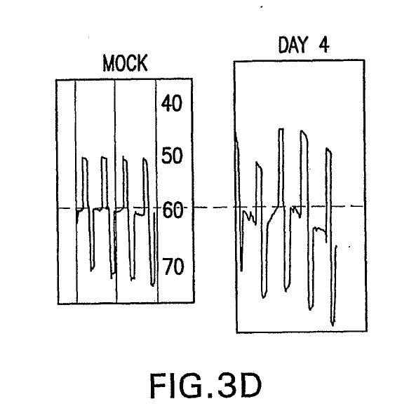

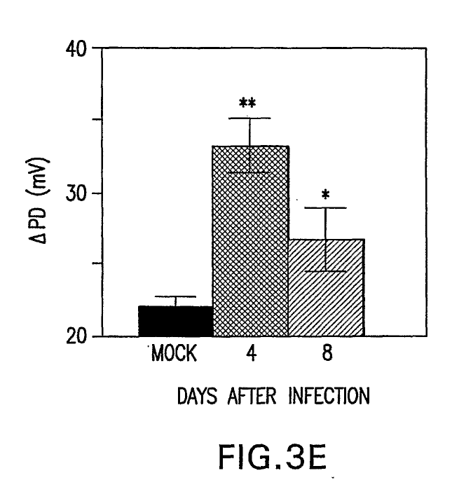

Effects of RSV infection on nasal potential difference. Infection with RSV for 2 days had no effect on basal NPD (nasal potential difference) or NPDAMIL (Amilori 6 -sensitive component of nasal potential difference) in BALB/c mice, as compared to mock-infected animals. However, basal NPD and NPDAMIL were significantly reduced at d4 and d8 (FIG. 3A-3C).

As an estimate of NRte (nasal transepitalial resistance) following RSV infection, the change in NPD (ΔNPD) elicited by applying a ±60 nA pulse to the nasal epithelium was measured. ΔNPD was significantly greater at d4 and d8 than in mock-infected controls (FIG. 3D-3E).

Effect of RSV infection and nucleotide synthesis inhibition on BALF nucleotides. BALF from uninfected mice contained equivalent levels of ATP and UTP, which were not affected by mock infection. However, RSV infection resulted in a doubling of UTP and ATP levels at d2, without a concomitant increase in BALF heme content (7.3±1.4 μM at d2, vs. 7.3±0.7 μM in uninfected mice). BALF nucleotides returned to control levels at d6 (Table 1).

No increase in BALF nucleotide levels was detected at d2 in leflunomide-treated, RSV-infected mice. In fact, leflunomide treatment reduced BALF content of both nucleotides to levels below those in untreated, uninfected mice (Table 1). Concomitant uridine treatment not only reversed the effect of leflunomide on BALF UTP and ATP levels but also caused a significant increase in the BALF nucleotide content over that in untreated RSV-infected mice.

TABLE 1
Effect of RSV infection and nucleotide synthesis inhibition
on BALF nucleotide levels.

| | n^{A} | ATP^{B} | UTP^{B} |
|---|---|---|---|
| Uninfected | 11 | 16 ± 2 | 16 ± 4 |
| Mock | 6 | 13 ± 4 | 10 ± 4 |
| d2 | 14 | 38 ± 7*** | 32 ± 4** |
| d6 | 9 | 17 ± 2 | 11 ± 4 |
| d2 LEF^{C} | 9 | 6 ± 1** | 5 ± 2*** |
| d2 LEF + U^{D} | 7 | 69 ± 29 | 95 ± 29** |

^{A}Number of mice per group in which nucleotide levels were evaluated
^{B}Mean nucleotide concentration in BALF ± SE (nmol/l)
^{C}Leflunomide-treated mice
^{D}Leflunomide- and uridine-treated mice
**p < 0.005,
***p < 0.0005, compared with uninfected mice

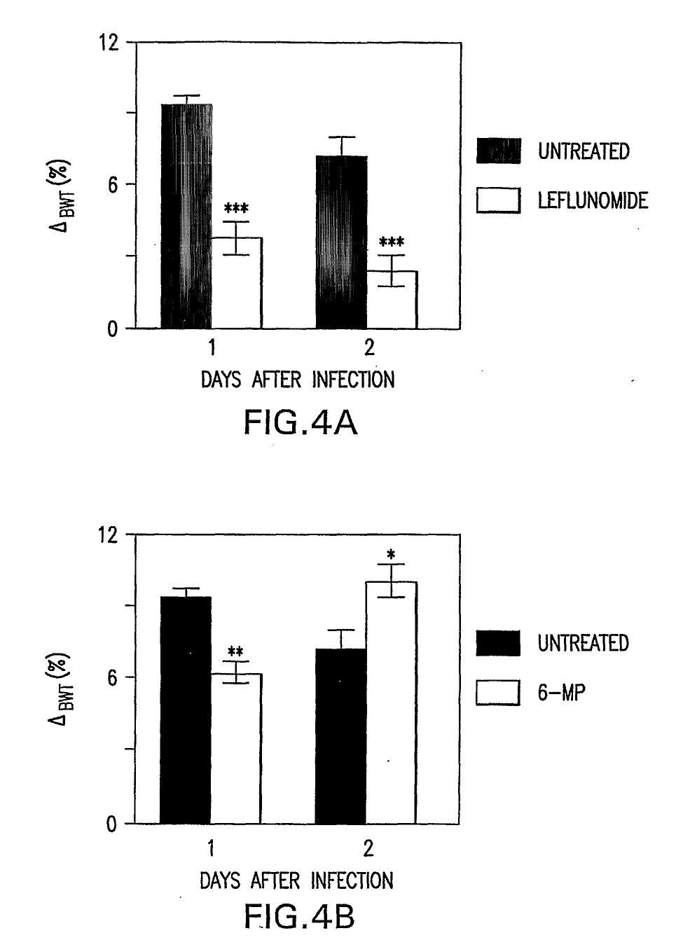

Effect of nucleotide synthesis inhibition on mouse body weight. During the pretreatment period, leflunomide caused no significant decline in body weight, as compared to methylcellulose-treated or untreated mice. More importantly, during the infection period, leflunomide therapy significantly reduced the degree of weight loss normally seen in BALB/c mice at D1 and d2 (FIG. 4). Concomitant administration of uridine throughout the leflunomide treatment period did not prevent this effect.

In contrast to this finding, treatment with 6-MP resulted in significant loss of body weight throughout the preinfection period, poor tolerance to anesthesia (resulting in sporadic deaths), and a significant increase in body weight loss at D1 and d2, as compared with both untreated, RSV-infected mice, and leflunomide-treated, RSV-infected mice (FIG. 4B).

Effect of nucleotide synthesis inhibition on RSV-mediated inhibition of AFC. Leflunomide pretreatment of RSV-infected mice blocked RSV-induced inhibition of AFC at d2 (Table 2). This effect was not mimicked by gavage with methylcellulose alone, and was reversed by concomitant uridine treatment. Uridine treatment alone had no effect on AFC. Leflunomide treatment also resulted in restoration of normal amiloride sensitivity to AFC: 57% of AFC in leflunomide-treated mice at d2 was amiloride-sensitive, compared to 61% in uninfected mice and −8% in untreated mice at d2.

In contrast to the beneficial effect of leflunomide therapy, a similar regimen of systemic pretreatment with the de novo purine synthesis inhibitor 6-mercaptopurine (6-MP) had no effect on AFC at d2 (Table 2). Finally, treatment of uninfected mice with leflunomide resulted in significant inhibition of AFC.

TABLE 2
Effect of nucleotide synthesis inhibition on RSV-mediated
inhibition of AFC at d2.

| | n^{A} | AFC^{B} |
|---|---|---|
| Uninfected | 7 | 34.89 ± 2.49*** |
| Uninfected AMIL^{C} | 7 | 14.65 ± 1.59††† |
| Uninfected LEF^{D} | 17 | 29.98 ± 1.5** |
| d2 | 25 | 22.01 ± 1.04 |
| d2 AMIL | 7 | 22.82 ± 1.92 |
| d2 MC^{E} | 11 | 22.89 ± 1.27 |
| d2 LEF | 14 | 34.52 ± 2.1*** |
| d2 LEF + AMIL | 11 | 14.92 ± 2.3††† |
| d2 U^{F} | 19 | 22.89 ± 2.22 |
| d2 LEF + U^{G} | 10 | 21.9 ± 2.69 |
| d2 6-MP^{H} | 11 | 16.52 ± 2.51 |

^{A}Number of mice in which AFC was evaluated
^{B}Mean % AFC ± SE
^{C}AFC with 1.5 mM amiloride added to the instillate
^{D}Leflunomide-treated mice
^{E}Methylcellulose-treated mice
^{F}Uridine-treated mice
^{G}Leflunomide- and uridine-treated mice
^{H}6-mercaptopurine-treated mice
**p < 0.005,
***p < 0.0005, compared with AFC at d2
†††p < 0.0005, compared with AFC at d2 AMIL

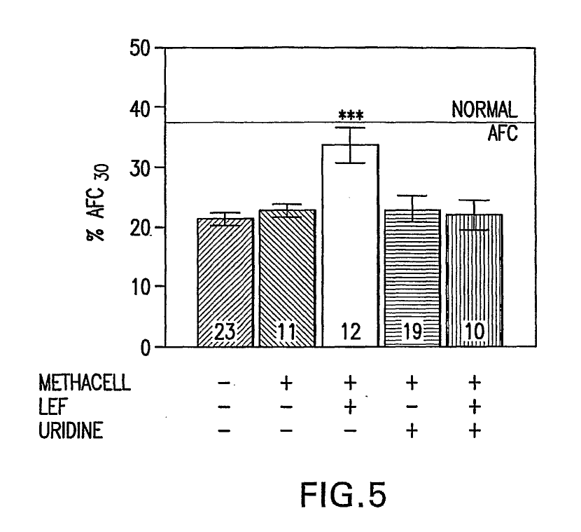

To systemically block de novo pyrimidine synthesis, mice were gavaged once daily for 8 days with 300 ml/mouse of the dihydro-orotate reductase (DHOR) inhibitor leflunomide (5 mg/kg suspended in 1% methylcellulose) or vehicle prior to infection, then at 0 and 24 hours p.i. AFC studies were performed at 48 hours p.i., with no additions to the AFC instillate.

Where indicated, attempts were made to reverse the effects of leflunomide (LEF) by concomitant administration of uridine throughout the leflunomide treatment period (1 mg/kg I.P., q12h). As shown in FIG. 5, gavage of mice with leflunomide (LEF) reversed RSV-mediated inhibition of AFC at day 2 p.i. The effect of LEF is prevented by concomitant administration of uridine. LEF had no effect on AFC in normal mice.

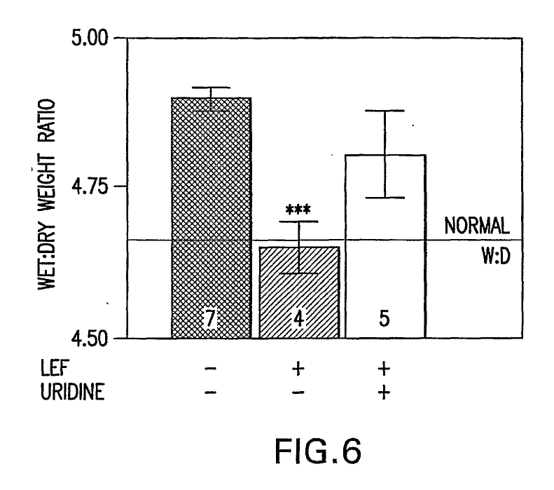

LEF treatment also resulted in a significant reduction in weight loss at days 1 and 2 p.i. and in bronchoalveolar lavage proinflammatory cytokine (IFN-a, Il-1b, TNF-a, KC) concentrations. As shown in FIG. 6, gavage of mice with leflunomide (LEF) reversed RSV-induced increased in lung water content at day 2 p.i. The effect of leflunomide was prevented by concomitant administration of uridine. Importantly, treatment of mice with LEF and/or uridine had no effect on virus replication in lung tissue at day 2 p.i..

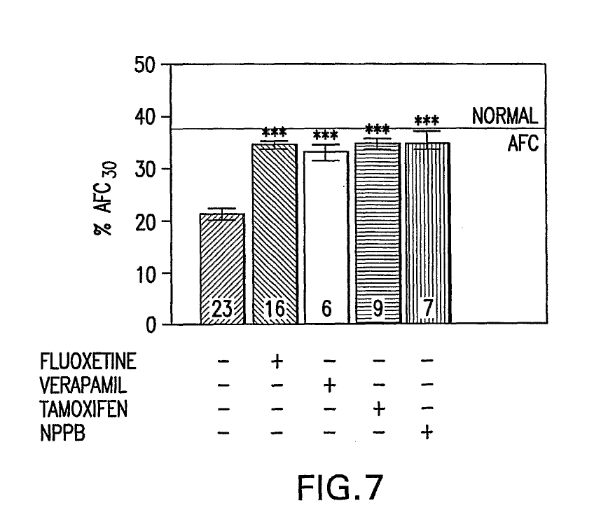

As shown in FIG. 7, addition of a wide spectrum of inhibitors of volume-regulated anion channels (VRACs) to the AFC instillate reversed RSV mediated inhibition of AFC at day 2 p.i. While some inhibitors used also had disparate effects on a variety of other cellular functions, these agents have only VRAC inhibition as a common effect. However, NPPB (100 mM) is relatively VRAC-specific. This finding demonstrated that UTP is released from cells via VRACs during early RSV infection. Fluoxetine (10 mM) also acts as a selective serotonin reuptake inhibitor. Verapamil (10 mM) also acts as a Ca⁺⁺ channel blocker. Tamoxifen (25 mM) is also an anti-estrogen.

Respiratory syncytial virus inhibits amiloride-sensitive AFC (indicative of active Na⁺ transport) at early timepoints after infection in a BALB/c mouse model, without inducing significant respiratory epithelial cytopathology. Moreover, inhibitory effects of RSV on AFC are mediated by UTP, through its action on P2Y purinergic receptors in the lung.

The UTP which mediates RSV-induced inhibition of AFC at day 2 after infection is derived from de novo synthesis, and inhibition of this pathway prevents RSV-induced reductions in AFC and increases in lung water content without altering viral replication. Furthermore, the UTP which mediates RSV-induced inhibition of AFC at day 2 after infection is released via volume-regulated anion channels.

Effects of leflunomide on RSV-induced inhibition of AFC at day 2 p.i. were demonstrated. Mice were pretreated for 8 days with leflunomide (5 mg/kg, suspended in 1% methylcellulose, once daily) by oral gavage, then infected with RSV and treated with leflunomide again at 24 hours p.i. This regimen prevented RSV-induced inhibition of AFC at day 2 p.i. The effect was not mimicked by gavage with methylcellulose alone, and was reversed by concomitant administration of uridine throughout the leflunomide treatment period (1 mg/kg i.p. q12h for 10 days). Again, uridine treatment alone had no effect on AFC. Leflunomide treatment also had no detrimental effect on AFC in normal (mock-infected) mice.

TABLE 3
Effects of leflunomide on RSV-induced inhibition of AFC at day 2 p.i.

| Treatment | n^{A} | AFC_{30BASAL}^{B} |
|---|---|---|
| None | 23 | 21.19 ± 0.94 |
| Methylcellulose | 11 | 22.89 ± 1.27 |
| Leflunomide | 12 | 33.4 ± 3.00*** |
| Uridine | 19 | 22.89 ± 2.22 |
| Leflunomide + uridine | 10 | 21.9 ± 2.69 |
| Leflunomide (mock-infected mice) | 7 | 33.16 ± 2.40*** |

^{A}Number of mice in which AFC was evaluated;
^{B}Mean % basal AFC after 30 minutes ± SE;
***p < 0.0005 (relative to untreated mice).
AFC_{30BASAL} in mock-infected BALB/c mice is 37.21 ± 1.2% (n = 8).

The inhibitory effect of leflunomide was not simply a result of an antiviral effect. Viral replication was unaffected by methylcellulose, leflunomide, or uridine treatment. Therefore, abrogation of RSV-induced inhibition of AFC was not a simple consequence of preventing viral replication, but was a result of a specific inhibitory effect of leflunomide on de novo pyrimidine synthesis.

Leflunomide therapy was associated with a normalization of lung wet:dry weight ratios (an index of lung water content and edema formation), which are increased at day 2 after RSV infection. Concomitant uridine treatment reversed this effect and resulted in increased wet:dry ratios (compared to mock-infected mice). Leflunomide therapy significantly reduced the degree of weight loss normally seen in BALB/c mice at days 1 and 2 p.i., suggesting a beneficial effect on appetite (possibly related to anti-inflammatory effects). This also suggested very limited leflunomide toxicity at this dose. Leflunomide therapy improved mean blood O₂ saturation at day 2 p.i., when a degree of hypoxemia is normally evident. Leflunomide therapy significantly reduced bronchoalveolar lavage proinflammatory cytokine (interferon-α, interleukin-1β, KC [the murine homolog of human interleukin-8] and tumor necrosis factor-α) levels, an effect that was only partially reversed by concomitant uridine therapy (and which may therefore partly be a consequence of nonspecific tyrosine kinase inhibition by the drug).

Taken together, these data demonstrated that leflunomide has several beneficial effects on RSV disease, without having direct antiviral effects. These effects included abrogation of hypoxemia and pulmonary edema, improvements in body weight, and reductions in pulmonary inflammation.

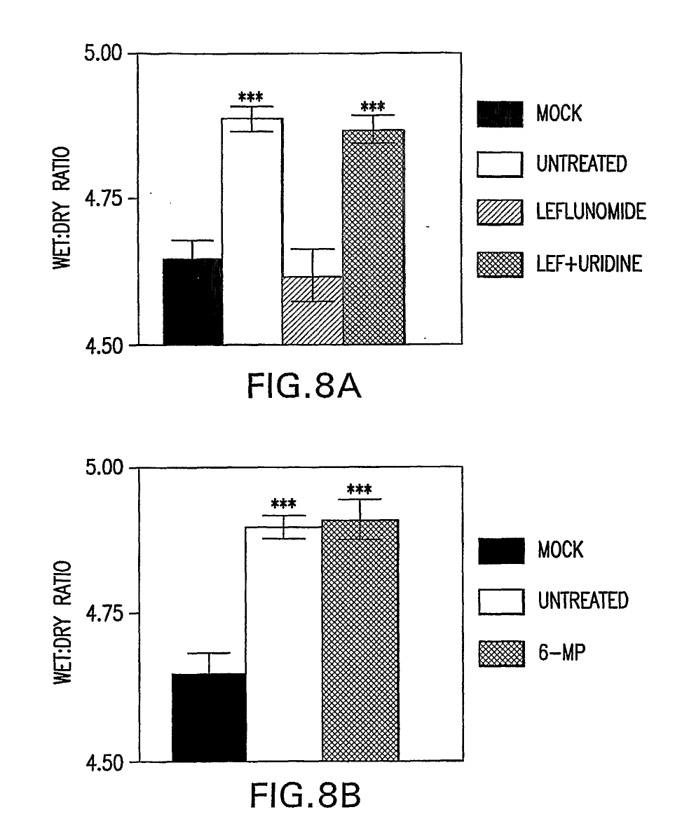

Effect of nucleotide synthesis inhibition on lung water content. Systemic therapy with leflunomide restored normal lung wet:dry weight ratios at day 2, while concomitant administration of uridine throughout the leflunomide treatment period prevented this effect (FIG. 8A). However, systemic therapy with 6-MP, which had no beneficial effect on AFC at day 2, did not alter lung wet:dry weight ratios at day 2 (FIG. 8B).

Effect of nucleotide synthesis inhibition on proinflammatory cytokines. Leflunomide is used clinically as an immunosuppressive agent. To verify efficacy of the treatment regimen, its effect on levels of proinflammatory cytokines in BALF was analyzed. No IL-4 or IL-10 was detectable at any timepoint after infection. Only small amounts of IL-1β and KC (the murine homolog of human IL-8) were detected in BALF from mock-infected mice (Table 4). Significant amounts of all other cytokines except IFN-γ were present at d2, but levels of IL-1β, KC, and TNF-γ declined at d4-d8. Significant quantities of IFN-γ were only found in BALF at d6 and d8.

Leflunomide therapy significantly reduced levels of IFN-α, IL-1β, KC, and TNF-α in BALF at d2 (Table 4). With the exception of IFN-α, this effect was only partially reversed by concomitant uridine therapy.

Importantly, 6-MP therapy resulted in a comparable decline in BALF IFN-α, IL-1β, KC, and TNF-α levels to that caused by leflunomide therapy (Table 4). There were no significant differences between IFN-α, IL-1β, KC, and TNF-α levels in mice treated with either agent at d2.

TABLE 4
Effect of RSV infection and nucleotide synthesis inhibition on BALF
proinflammatory cytokines.

| | n^{A} | IFN-α^{B} | IFN-γ^{B} | IL-1β^{B} | KC^{B} | TNF-α^{B} |
|---|---|---|---|---|---|---|
| Mock | 8 | 0*** | 0 | 10 ± 6*** | 80 ± 21*** | 0*** |
| d2 | 13 | 151 ± 12 | 2 ± 1 | 136 ± 24 | 913 ± 36 | 81 ± 16 |
| d4 | 8 | ND^{F} | 30 ± 16 | 6 ± 1*** | 106 ± 27*** | 0*** |
| d6 | 8 | ND | 912 ± 116*** | 20 ± 3*** | 72 ± 9*** | 1 ± 1*** |
| d8 | 6 | ND | 195 ± 25*** | 8 ± 2*** | 88 ± 16*** | 0*** |
| d2 LEF^{C} | 12 | 72 ± 12*** | ND | 20 ± 8*** | 425 ± 58*** | 0*** |
| d2 LEF + U^{D} | 10 | 246 ± 68 | ND | 20 ± 6*** | 334 ± 62*** | 0*** |
| d2 6-MP^{E} | 8 | 105 ± 14* | ND | 9 ± 3*** | 329 ± 63*** | 0*** |

^{A}Number of mice in which BALF cytokine levels were measured
^{B}Mean cytokine concentration in BALF ± SE (pg/ml)
^{C}Leflunomide-treated mice
^{D}Leflunomide- and uridine-treated mice
^{E}6-mercaptopurine-treated mice
^{F}Not done
***p < 0.0005, compared with levels at d2

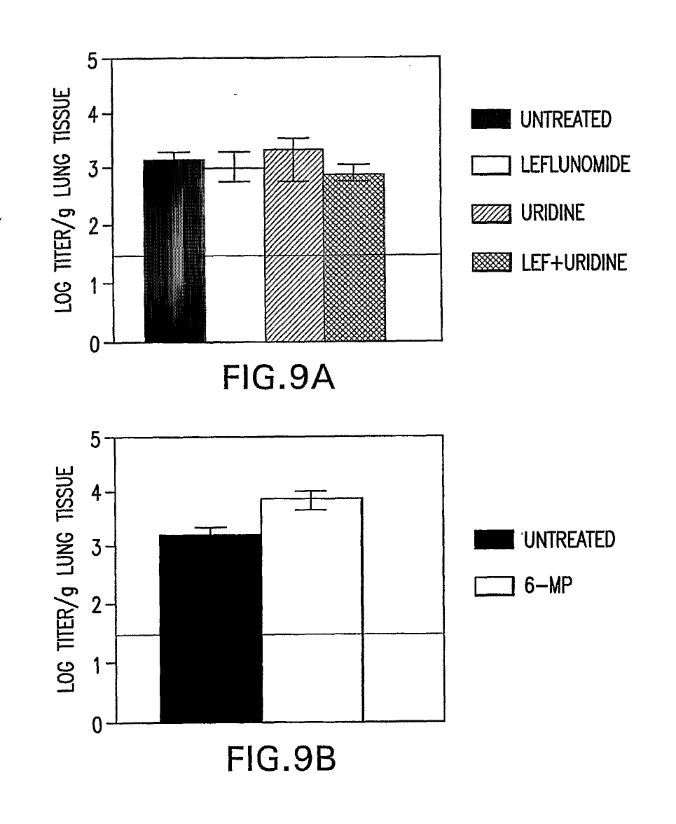

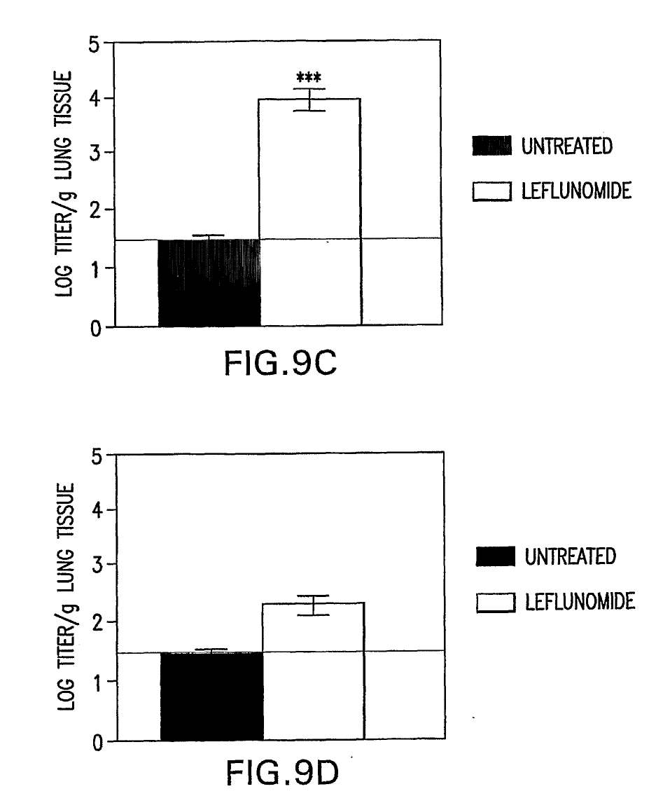

Effect of nucleotide synthesis inhibition on RSV replication in mouse lungs. Viral replication at d2 was unaffected by either leflunomide or uridine treatment (FIG. 9A). Likewise, 6-MP pretreatment had no significant inhibitory effect on virus replication in mouse lungs at d2 (FIG. 9B). When leflunomide treatment was continued throughout the 8 day infection period, virus replication persisted at high levels at d8 (FIG. 9C). However, when leflunomide treatment was discontinued after d2, viral replication was only minimally increased at d8 (FIG. 9D).

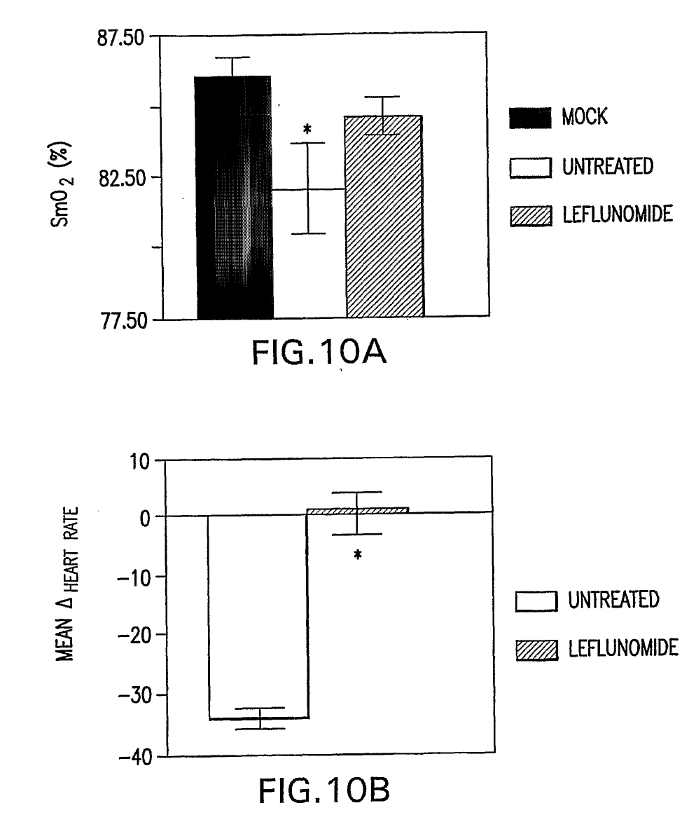

Effect of leflunomide therapy on hypoxemia after RSV infection. Leflunomide therapy resulted in a normalization of SmO₂ readings at d2 (FIG. 10A). Likewise, leflunomide therapy prevented the increase in % ΔHR30 seen in RSV-infected mice during AFC procedures at d2 (FIG. 10B).

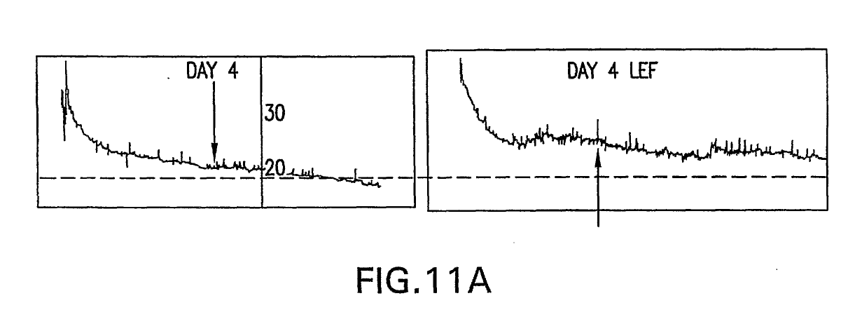

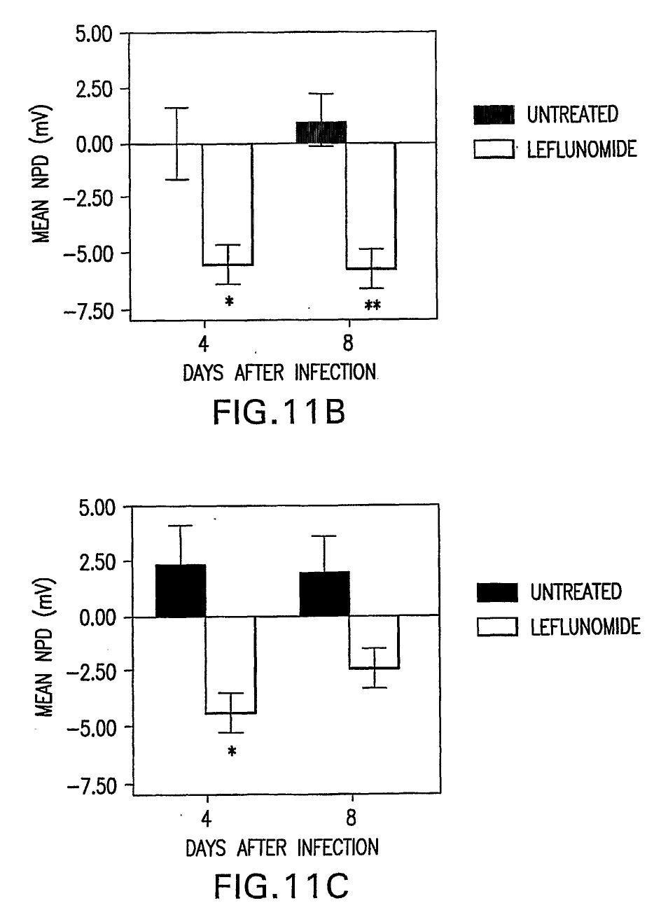

Effects of leflunomide therapy on nasal potential difference after RSV infection. Treatment with leflunomide throughout the infection period completely prevented RSV-induced declines in basal NPD and NPDAMIL (FIG. 11A-11C).

Effect of anion channel blockade on RSV-mediated inhibition of AFC. RSV-mediated inhibition of AFC at d2 was blocked by addition to the AFC instillate of each of several structurally unrelated VRAC inhibitors: fluoxetine, tamoxifen, clomiphene, verapamil, NPPB, or IAA-94 (Table 5). This effect was reversed by concomitant addition of 500 nM UTP to the instillate. In contrast, AFC at d2 was unaffected by inhibition of cystic fibrosis transmembrane regulator and Ca2+-activated C1-channel activity, with glibenclamide and niflumic acid, respectively.

TABLE 5
Effect of addition of anion channel inhibitors to the AFC instillate on
RSV-mediated inhibition of AFC at d2.

| | Concentration | | |
|---|---|---|---|
| Inhibitor | (μM) | n^{A} | AFC^{B} |
| None | — | 25 | 22.01 ± 1.04 |
| Fluoxetine | 10 | 16 | 34.54 ± 0.79*** |
| Fluoxetine + UTP | 10/0.5 | 8 | 23.64 ± 2.42 |
| Tamoxifen | 25 | 9 | 34.50 ± 0.94*** |
| Clomiphene | 20 | 8 | 31.05 ± 2.65*** |
| Verapamil | 10 | 6 | 33.04 ± 1.49** |
| NPPB^{C} | 100 | 9 | 32.70 ± 2.18** |
| R(+)-IAA 94^{D} | 100 | 5 | 34.25 ± 1.98*** |
| Glibenclamide | 100 | 9 | 24.24 ± 4.24 |
| Niflumic acid | 100 | 10 | 20.28 ± 1.53 |

^{A}Number of mice in which AFC was evaluated
^{B}Mean % AFC ± SE
^{C}5-nitro-2-(3-phenylpropylamino) benzoic acid
^{D}R(+)-[(6,7-dichloro-2-cyclopentyl-2,3-dihydro-2-methyl-1-oxo-1H-inden-5yl)-oxy] acetic acid 94
**p < 0.005,
***p < 0.0005, compared with AFC_{30BASAL} at d2

Effects of A77-1726 of RSV-mediated inhibition of AFC. As previously known, intranasal infection of BALB/c mice with respiratory syncytial virus (RSV) strain A2 resulted in reduced basal and amiloride-sensitive AFC at days 2 and 4 post-infection (p.i.), and this inhibition was mediated by UTP, acting via P2Y receptors (AJPLCMP, 2004). RSV-mediated inhibition of AFC at day 2 p.i. has been further shown to be prevented by addition to the AFC instillate of 25 mM A77-1726, which blocked de novo pyrimidine synthesis, but not by either 25 mM mycophenolic acid or 6-mercaptopurine, both of which block de novo purine synthesis. A77-1726-mediated block was reversed by addition of 50 mM uridine (which allows pyrimidine synthesis via the salvage pathway) and not recapitulated by 25 mM genistein (which mimics the nonspecific tyrosine kinase inhibitor effects of A77-1726), indicating that the blocking effect of A77-1726 was mediated through the de novo pyrimidine synthesis pathway. Similarly, treatment of mice with the de novo pyrimidine synthesis inhibitor leflunomide (5 mg/kg p.o. in 1% methylcellulose for 10 days) reversed the inhibitory effect of RSV on AFC. Moreover, inhibitors of volume-regulated anion channel (VRAC) function, such as fluoxetine (10 mM), verapamil (10 mM), and tamoxifen (25 mM) also blocked RSV-mediated inhibition of AFC at day 2 p.i. Together, these data demonstrated that the UTP that inhibits AFC during RSV infection is both derived from de novo pyrimidine synthesis and released via VRACs. These pathways offer novel therapeutic approaches to prevent UTP-induced reductions in AFC, which contribute to formation of an increased volume of fluid mucus, airway congestion, and rhinorrhea following RSV infection.

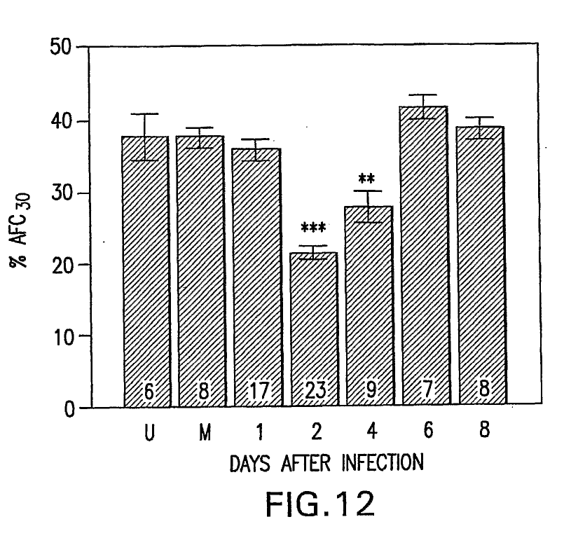

As shown in FIG. 12, infection with RSV significantly inhibits basal alveolar fluid clearance (AFC) at days 2 and 4 post infection (p.i.). Mock infection (M) has no effect on AFC, compared to uninfected mice (U). Basal AFC was inhibited by 43% (from mock-infected values) at day 2 and by 26% at day 4. Amiloride sensitivity of AFC was also reduced at day 1, and absent at days 2 and 4 p.i..

RSV-mediated inhibition of AFC at day 2 p.i. was reversed by addition of apyrase (which degrades both UTP and ATP), or UDP-glucose pyrophosphorylase (which degrades UTP in the presence of glucose-1-phosphate and inorganic pyrophosphatase) to the AFC instillate, but not by addition of hexokinase (which degrades ATP in the presence of glucose). Addition of a P2Y receptor-specific antagonist (200 mM XAMR0721) to the instillate also reversed RSV-mediated inhibition of AFC at day 2 p.i.

AFC studies were performed on anesthetized, ventilated BALB/c mice with normal body temperature and blood gases, over a 30 minute period after intratracheal instillation of 0.3 ml of isosmolar NaCl containing 5% fatty acid-free BSA. The number of mice analyzed per group is listed in the relevant bar of each graph.

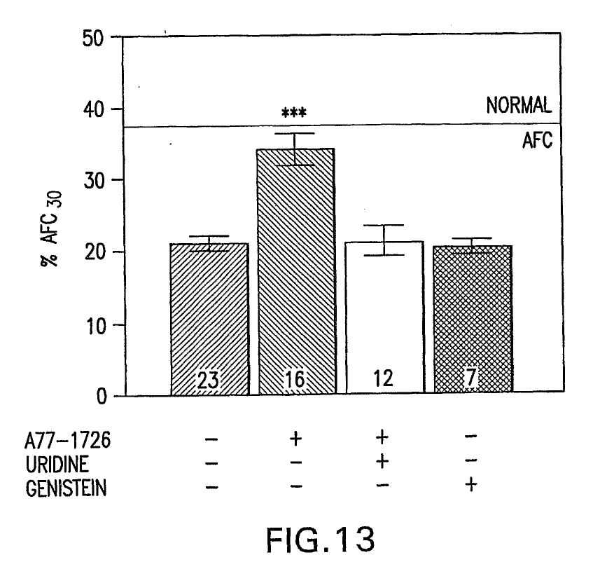

As shown in FIG. 13, addition of an inhibitor of dihydro-orotate reductase (25 μm A77-1726) to the AFC instillate reversed RSV-mediated inhibition of AFC at day 2 p.i. The effect of A77-1726 was fully reversed by concomitant addition of 50 mM uridine to the AFC instillate, but is not recapitulated by 25 mM genistein (a nonspecific tyrosine kinase inhibitor). Thus, the effect of A77-1726 is specific to the de novo pyrimidine synthesis pathway.

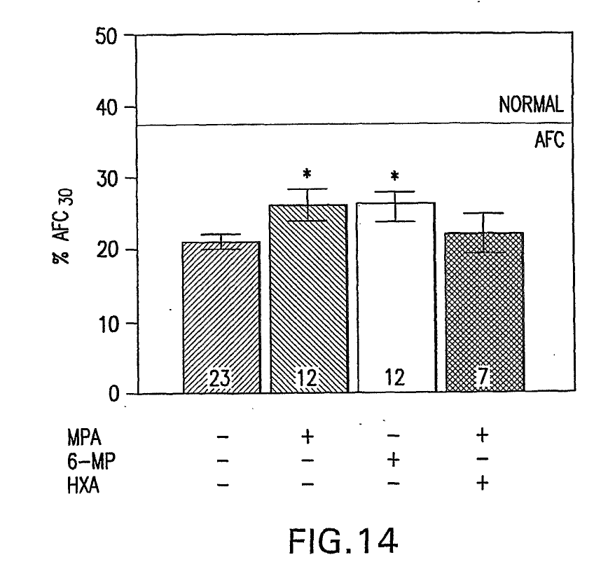

As shown in FIG. 14, addition of inhibitors of IMP dehydrogenase (25 μm 6-MP or MPA) to the AFC instillate had only a minor effect on RSV-mediated inhibition of AFC at day 2 p.i. The small effect of IMP dehydrogenase inhibitors was a consequence of depletion of ATP, which is a necessary precursor for de novo pyrimidine synthesis. The MPA effect was fully reversed by concomitant addition of 50 mM hypoxanthine (HXA) to the AFC instillate, allowing synthesis of ATP via the purine salvage pathway. This finding demonstrated that ATP reserves are low during RSV infection

RSV-induced inhibition of AFC at day 2 p.i. is prevented by A77-1726, an inhibitor of de novo pyrimidine synthesis. The effect of A77-1726 was blocked by addition of exogenous uridine, which promotes UTP synthesis via the salvage pathway, and is not replicated by genistein, which mimics the nonspecific tyrosine kinase inhibitory effects of A77-1726. Inhibitors of de novo purine synthesis, such as mycophenolic acid (MPA) and 6-mercaptopurine (6-MP), had only a small blocking effect on RSV-mediated inhibition of AFC, probably as a consequence of reduced ATP synthesis (ATP is a necessary precursor for de novo pyrimidine synthesis). Again, this effect was blocked by addition of exogenous hypoxanthine, which promotes ATP synthesis via the salvage pathway. Interestingly, RSV-induced inhibition of AFC at day 2 p.i. was also prevented by a variety of different inhibitors of volume-regulated anion channels (VRACs), which have been proposed as a release mechanism for ATP and UTP, and by inhibition of Rho kinase, which is known to be activated by RSV and also known to activate VRACs.

TABLE 6
Effects of inhibitors of pyrimidine and purine synthesis and
VRACs on RSV-induced inhibition of AFC at day 2 p.i.

| Target | Inhibitor | Conc.^{A} | n^{B} | AFC_{30BASAL}^{C} |
|---|---|---|---|---|
| | None | — | 23 | 21.19 ± 0.94 |
| De novo | A77-1726 | 25 | 16 | 34.06 ± 1.88*** |
| pyrimidine | A77-1726 + Uridine | 25/50 | 12 | 21.3 ± 2.02 |
| synthesis | | | | |
| Tyrosine kinases | Genistein | 25 | 7 | 20.39 ± 0.73 |
| De novo purine | MPA | 25 | 12 | 26.2 ± 1.7* |
| synthesis | 6-MP | 25 | 12 | 26.31 ± 1.85* |
| | MPA + | 25/50 | 7 | 22.36 ± 2.73 |
| | Hypoxanthine | | | |
| VRACs | Fluoxetine | 10 | 16 | 34.54 ± 0.79*** |
| | Verapamil | 10 | 6 | 33.04 ± 1.49*** |
| | Tamoxifen | 25 | 9 | 34.5 ± 0.95*** |
| | Clomiphene | 20 | 8 | 31.0 ± 2.67** |
| | NPPB | 100 | 7 | 35.12 ± 1.94*** |
| Rho kinases | ROCK | 20 | 10 | 36.58 ± 2.11*** |
| | inhibitor | | | |

^{A}Final concentration (μM);
^{B}Number of mice in which AFC was evaluated;
^{C}Mean % basal AFC after 30 minutes ± SE;
*p < 0.05;
**p < 0.005;
***p < 0.0005 (all relative to untreated mice).
AFC_{30BASAL} in mock-infected BALB/c mice is 37.21 ± 1.2% (n = 8).

Post-infection A77-1726 treatment on RSV-induced inhibition of AFC at day 2 p.i. When mice were treated at 24 hours p.i. by intranasal administration of A77-1726 (50 μM in 100 μl normal saline, divided between both nostrils), the inhibitory effect of RSV on AFC at 24 hours p.i. was completely blocked, demonstrating that, when administered topically into the lungs, A77-1726 had a prolonged inhibitory effect on de novo pyrimidine synthesis. A77-1726 intranasal pretreatment was also associated with a normalization of lung wet:dry weight ratios (an index of lung water content and edema formation), which are increased at day after RSV infection.

TABLE 7
Effects of intranasal A77-1726 treatment at 24 hours p.i. on
RSV-induced inhibition of AFC at day 2 p.i.

| Infection status | Treatment | n^{A} | AFC_{30BASAL}^{B} |
|---|---|---|---|
| Uninfected | None | 7 | 34.9 ± 2.5 |
| Uninfected | A77-1726^{C} | 10 | 23.19 ± 5.95*** |
| RSV-day 2 p.i. | None | 23 | 21.19 ± 0.94 |
| RSV-day 2 p.i. | A77-1726^{D} | 14 | 32.68 ± 1.1*** |

^{A}Number of mice in which AFC was evaluated;
^{B}Mean % basal AFC after 30 minutes ± SE;
^{C}50 μM in 100 μl normal saline, administered intranasally 24 hours prior to AFC assay;
^{D}50 μM in 100 μl normal saline, administered intranasally 24 hours after infection;
***p < 0.0005 (relative to untreated mice).

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

### REFERENCES

1. Sartori, C. and Matthay, M. A. 2002. Alveolar epithelial fluid transport in acute lung injury: new insights. Eur. Respir. J. 20:1299-1313.

2. Ware, L. B. and Matthay, M. A. 2001. Alveolar fluid clearance is impaired in the majority of patients with acute lung injury and the acute respiratory distress syndrome. Am. J. Respir. Crit Care Med. 163:1376-1383.

3. Black, C. P. 2003. Systematic review of the biology and medical management of respiratory syncytial virus infection. Respir. Care 48:209-233.

4. Davis, I. C., Sullender, W. M., Hiclman-Davis, J. M., Lindsey, J. R., and Matalon, S. 2004. Nucleotide-mediated inhibition of alveolar fluid clearance in BALB/c mice after respiratory syncytial virus infection. Am. J. Physiol Lung Cell Mol. Physiol 286:L112-L120.

5. Smee, D. F., Bailey, K. W., Wong, M. H., and Sidwell, R. W. 2001. Effects of cidofovir on the pathogenesis of a lethal vaccinia virus respiratory infection in mice. Antiviral Res. 52:55-62.

6. Grubb, B. R., Vick, R. N., and Boucher, R. C. 1994. Hyperabsorption of Na+ and raised Ca(2+)-mediated C1-secretion in nasal epithelia of CF mice. Am. J. Physiol 266:C1478-C1483.

7. Lazarowski, E. R. and Harden, T. K. 1999. Quantitation of extracellular UTP using a sensitive enzymatic assay. Br. J. Pharmacol. 127:1272-1278.

8. Pinschewer, D. D., Ochsenbein, A. F., Fehr, T., and Zinkernagel, R. M. 2001. Leflunomide-mediated suppression of antiviral antibody and T cell responses: differential restoration by uridine. Transplantation 72:712-719.

9. Krynetskaia, N. F., Brenner, T. L., Krynetski, E. Y., Du, W., Panetta, J. C., Ching-Hon, P., and Evans, W. E. 2003. Msh2 deficiency attenuates but does not abolish thiopurine hematopoietic toxicity in msh2−/− mice. Mol. Pharmacol. 64:456-465.

10. Nilius, B., Eggermont, J., and Droogmans, G. 2000. The endothelial volume-regulated anion channel, VRAC. Cell Physiol Biochem. 10:313-320.

11. Sheppard, D. N. and Robinson, K. A. 1997. Mechanism of glibenclamide inhibition of cystic fibrosis transmembrane conductance regulator C1-channels expressed in a murine cell line. J. Physiol 503 (Pt 2):333-346.

12. White, M. M. and Aylwin, M. 1990. Niflumic and flufenamic acids are potent reversible blockers of Ca2(+)-activated C1-channels in Xenopus oocytes. Mol. Pharmacol. 37:720-724.

13. Huang, M., Bigos, D., and Levine, M. 1998. Ventricular arrhytlmuia associated with respiratory syncytial viral infection. Pediatr. Cardiol. 19:498-500.

14. Eisenhut, M., Sidaras, D., Johnson, R., Newland, P., and Thorburn, K. 2004. Cardiac Troponin T levels and myocardial involvement in children with severe respiratory syncytial virus lung disease. Acta Paediatr. 93:887-890.

15. Matalon, S., Hardiman, K. M., Jain, L., Eaton, D. C., Kotlikoff, M., Eu, J. P., Sun, J., Meissner, G., and Stamler, J. S. 2003. Regulation of ion channel structure and function by reactive oxygen-nitrogen species. Am. J. Physiol Lung Cell Mol. Physiol 285 :L1184-L1189.

16. Knowles, M. R., Paradiso, A. M., and Boucher, R. C. 1995. In vivo nasal potential difference: techniques and protocols for assessing efficacy of gene transfer in cystic fibrosis. Hum. Gene Ther. 6:445-455.

17. Alton, E. W., Currie, D., Logan-Sinclair, R., Warner, J. O., Hodson, M. E., and Geddes, D. M. 1990. Nasal potential difference: a clinical diagnostic test for cystic fibrosis. Eur. Respir. J. 3:922-926.

18. Hofmann, T., Bohmer, O., Huls, G., Terbrack, H. G., Bittner, P., Klingmuller, V., Heerd, E., and Lindemann, H. 1997. Conventional and modified nasal potential-difference measurement in cystic fibrosis. Am. J. Respir. Crit Care Med. 155:1908-1913.

19. Knowles, M. R., Carson, J. L., Collier, A. M., Gatzy, J. T., and Boucher, R. C. 1981. Measurements of nasal transepithelial electric potential differences in normal human subjects in vivo. Am. Rev. Respir. Dis. 124:484-490.

20. Wilson, R., Alton, E., Rutman, A., Higgins, P., Al Nalkib, W., Geddes, D. M., Tyrrell, D. A., and Cole, P. J. 1987. Upper respiratory tract viral infection and mucociliary clearance. Eur. J. Respir. Dis. 70:272-279.

21. Hardiman, K. M., McNicholas-Bevensee, C. M., Fortenberry, J., Myles, C. T., Malik, B., Eaton, D. C., and Matalon, S. 2004. Regulation of amiloride-sensitive Na(+) transport by basal nitric oxide. Am. J. Respir. Cell Mol.Biol 30:720-728.

22. Donaldson, S. H., Lazarowski, E. R., Picher, M., Knowles, M. R., Stutts, M. J., and Boucher, R. C. 2000. Basal nucleotide levels, release, and metabolism in normal and cystic fibrosis airways. Mol. Med. 6:969-982.

23. Lazarowski, E. R., Homolya, L., Boucher, R. C., and Harden, T. K. 1997. Direct demonstration of mechanically induced release of cellular UTP and its implication for uridine nucleotide receptor activation. J. Biol. Chem. 272:24348-24354.

24. Cressman, V. L., Lazarowski, E., Homolya, L., Boucher, R. C., Koller, B. H., and Grubb, B. R. 1999. Effect of loss of P2Y(2) receptor gene expression on nucleotide regulation of murine epithelial C1(−) transport. J. Biol. Chem. 274:26461-26468.

25. Lazarowski, E. R., Boucher, R. C., and Harden, T. K. 2003. Mechanisms of release of nucleotides and integration of their action as P2X- and P2Y-receptor activating molecules. Mol. Pharmacol. 64:785-795.

26. Okada, S. F., O'Neal, W. K., Huang, P., Nicholas, R. A., Ostrowski, L. E., Craigen, W. J., Lazarowski, E. R., and Boucher, R. C. 2004. Voltage-dependent anion channel-1 (VDAC-1) contributes to ATP release and cell volume regulation in murine cells. J. Gen. Physiol 124:513-526.

27. Fairbanks, L. D., Bofill, M., Ruckemann, K., and Simmonds, H. A. 1995. Importance of ribonucleotide availability to proliferating T-lymphocytes from healthy humans. Disproportionate expansion of pyrimidine pools and contrasting effects of de novo synthesis inhibitors. J. Biol. Chem. 270:29682-29689.

28. Di Virgilio, F., Chiozzi, P., Ferrari, D., Falzoni, S., Sanz, J. M., Morelli, A., Torboli, M., Bolognesi, G., and Baricordi, O. R. 2001. Nucleotide receptors: an emerging family of regulatory molecules in blood cells. Blood 97:587-600.

29. Davis, J. P., Cain, G. A., Pitts, W. J., Magolda, R. L., and Copeland, R. A. 1996. The immunosuppressive metabolite of leflunomide is a potent inhibitor of human dihydroorotate dehydrogenase. Biochemistry 35:1270-1273.

30. Huang, M. and Graves, L. M. 2003. De novo synthesis of pyrimidine nucleotides; emerging interfaces with signal transduction pathways. Cell Mol. Life Sci. 60:321-336.

31. Galietta, L. J., Folli, C., Marchetti, C., Romano, L., Carpani, D., Conese, M., and Zegarra-Moran, O. 2000. Modification of transepithelial ion transport in human cultured bronchial epithelial cells by interferon-gamma. Am. J. Physiol Lung Cell Mol. Physiol 278:L1186-L1194.

32. Rezaiguia, S., Garat, C., Delclaux, C., Meignan, M., Fleury, J., Legrand, P., Matthay, M. A., and Jayr, C. 1997. Acute bacterial pneumonia in rats increases alveolar epithelial fluid clearance by a tumor necrosis factor-alpha-dependent mechanism. J. Clin. Invest. 99:325-335.

33. Fukuda, N., Jayr, C., Lazrak, A., Wang, Y., Lucas, R., Matalon, S., and Matthay, M. A. 2001. Mechanisms of TNF-alpha stimulation of amiloride-sensitive sodium transport across alveolar epithelium. Am. J. Physiol Lung Cell Mol. Physiol 280:L1258-L1265.

34. Galietta, L. J., Pagesy, P., Folli, C., Caci, E., Romio, L., Costes, B., Nicolis, E., Cabrini, G., Goossens, M., Ravazzolo, R. et al. 2002. IL-4 is a potent modulator of ion transport in the human bronchial epithelium in vitro. J. Immunol. 168:839-845.

35. Xu, X., Shen, J., Mall, J. W., Myers, J. A., Huang, W., Blinder, L., Saclarides, T. J., Williams, J. W., and Chong, A. S. 1999. In vitro and in vivo antitumor activity of a novel immunomodulatory drug, leflunomide: mechanisms of action. Biochem. Pharmacol. 58:1405-1413.

36. Schlapfer, E., Fischer, M., Ott, P., and Speck, R. F. 2003. Anti-HIV-1 activity of leflunomide: a comparison with mycophenolic acid and hydroxyurea. AIDS 17:1613-1620.

37. Knight, D. A., Hejmanowski, A. Q., Dierksheide, J. E., Williams, J. W., Chong, A. S., and Waldman, W. J. 2001. Inhibition of herpes simplex virus type 1 by the experimental immunosuppressive agent leflunomide. Transplantation 71:170-174.

38. Waldman, W. J., Knight, D. A., Lurain, N. S., Miller, D. M., Sedmak, D. D., Williams, J. W., and Chong, A. S. 1999. Novel mechanism of inhibition of cytomegalovirus by the experimental immunosuppressive agent leflunomide. Transplantation 68:814-825.

39. Graham, B. S., Perkins, M. D., Wright, P. F., and Karzon, D. T. 1988. Primary respiratory syncytial virus infection in mice. J. Med. Virol. 26:153-162.

40. Rutigliano, J. A., Johnson, T. R., Hollinger, T. N., Fischer, J. E., Aung, S., and Graham, B. S. 2004. Treatment with anti-LFA-1 delays the Cd8+ cytotoxic-T-lymphocyte response and viral clearance in mice with primary respiratory syncytial virus infection. J. Virol. 78:3014-3023.

41. Rutigliano, J. A. and Graham, B. S. 2004. Prolonged production of TNF-alpha exacerbates illness during respiratory syncytial virus infection. J. Immunol. 173:3408-3417.

42. Schwiebert, E. M. 2001. ATP release mechanisms, ATP receptors and purinergic signalling along the nephron. Clin. Exp. Pharmacol. Physiol 28:340-350.

43. Huang, P., Lazarowski, E. R., Tarran, R., Milgram, S. L., Boucher, R. C., and Stutts, M. J. 2001. Compartmentalized autocrine signaling to cystic fibrosis transmembrane conductance regulator at the apical membrane of airway epithelial cells. Proc. Natl. Acad. Sci. U.S.A. 98:14120-14125.

44. Marriott, I., Inscho, E. W., and Bost, K. L. 1999. Extracellular uridine nucleotides initiate cytokine production by murine dendritic cells. Cell Immunol. 195:147-156.

45. Idzko, M., Panther, E., Bremer, H. C., Sorichter, S., Luttmann, W., Virchow, C. J., Jr., Di Virgilio, F., Herouy, Y., Norgauer, J., and Ferrari, D. 2003. Stimulation of P2 purinergic receptors induces the release of eosinophil cationic protein and interleukin-8 from human eosinophils. Br. J. Pharmacol. 138:1244-1250.

46. Mutlu, G. M., Koch, W. J., and Factor, P. 2004. Alveolar epithelial {beta} 2-adrenergic receptors: Their role in regulation of alveolar active sodium transport. Am. J. Respir. Crit Care Med. 170:1270-1275.

47. Rozman, B. 2002. Clinical pharmacokinetics of leflunomide. Clin. Pharmacokinet. 41:421-430.

1. A composition comprising a pyrimidine synthesis inhibitor and a pharmaceutically acceptable carrier, wherein the composition is suitable for topical administration to a pulmonary epithelial cell of a subject.

2. The composition of claim 1, wherein the composition is an inhalant.

3. The composition of claim 1, wherein the composition is aerosolized.

4. The composition of claim 1, wherein the composition is nebulized.

5. The composition of claim 1, wherein the pyrimidine synthesis inhibitor is leflunomide.

6. The composition of claim 1, wherein the pyrimidine synthesis inhibitor is A77-1726.

7. The composition of claim 1, wherein the pyrimidine synthesis inhibitor is an inhibitor of dihydro-orate reductase.

8. The composition of claim 1, wherein the composition is in a form suitable for intranasal administration.

9. (canceled)

10. (canceled)

11. A device comprising at least one metered dose of a composition comprising a therapeutic amount of a pyrimidine synthesis inhibitor wherein each metered dose comprises a therapeutic amount or a portion thereof of the pyrimidine synthesis inhibitor for treating a pulmonary disease in a subject.

12. The device of claim 11, wherein composition is in a form adaptable for topical administration to a pulmonary epithelial cell of a subject.

13. The device of claim 11, wherein the composition is an inhalant.

14. The device of claim 11, wherein the composition is aerosolized.

15. The device of claim 11, wherein the composition is nebulized.

16. The device of claim 11, wherein the pyrimidine synthesis inhibitor is leflunomide.

17. The device of claim 11, wherein the pyrimidine synthesis inhibitor is A77-1726.

18. The device of claim 11, wherein the pyrimidine synthesis inhibitor is an inhibitor of dihydro-orate reductase.

19. The device of claim 11, wherein the composition is in a form suitable for intranasal administration.

20. The device of claim 11, wherein the pulmonary disease is a respiratory syneytial virus infection.

21. A method of increasing Na⁺ dependent fluid clearance by a pulmonary epithelial cell comprising contacting the cell with an effective amount of a pyrimidine synthesis inhibitor, wherein the contacting causes increased Na⁺ dependent fluid clearance by the cell.

22. (canceled)

23. (canceled)

24. The method of claim 21, wherein the pyrimidine synthesis inhibitor is leflunomide.

25. The method of claim 21, wherein the pyrimidine synthesis inhibitor is A77-1726.

26. The method of claim 21, wherein the pyrimidine synthesis inhibitor is a dihydro-orate reductase inhibitor.

27. A method of treating a pulmonary disease in a subject comprising, contacting a plurality of pulmonary epithelial cells in the subject with an effective amount of a pyrimidine synthesis inhibitor, wherein the effective amount of the pyrimidine synthesis inhibitor causes increased Na⁺ dependent alveolar fluid clearance in the subject.

28. (canceled)

29. (canceled)

30. (canceled)

31. The method of claim 27, wherein the pyrimidine synthesis inhibitor comprises leflunomide.

32. The method of claim 27, wherein the pyrimidine synthesis inhibitor comprises A77-1726.

33. The method of claim 27, wherein the effective amount of a pyrimidine synthesis inhibitor comprises a dihydro-orate reductase inhibitor.

34. A method of reducing one or more symptoms or physical signs of a respiratory syncytial virus infection in a subject at risk for a respiratory syncytial virus infection comprising, administering to the subject a composition comprising an effective amount of a pyrimidine synthesis inhibitor.

35. The method of claim 34, wherein the pyrimidine synthesis inhibitor is an inhibitor of dihydro-oroate reductase.

36. The method of claim 34, wherein the pyrimidine synthesis inhibitor is leflunomide.

37. The method of claim 34, wherein the pyrimidine synthesis inhibitor is A77-1726.

38. A method comprising, identifying a subject at risk for respiratory syncytial virus infection and administering to the subject a composition comprising an effective amount of a pyrimidine synthesis inhibitor.

39. The method of claim 38, wherein the pyrimidine synthesis inhibitor is an inhibitor of dihydro-oroate reductase.

40. The method of claim 38, wherein the pyrimidine synthesis inhibitor is leflunomide.

41. The method of claim 38, wherein the pyrimidine synthesis inhibitor is A77-1726.

42. A method comprising, identifying a subject with a respiratory syncytial virus infection and administering to the subject a composition comprising a pyrimidine synthesis inhibitor in an amount effective to reduce Na⁺ dependent alveolar fluid in the subject.

43. (canceled)

44. (canceled)

45. (canceled)

46. (canceled)

47. A method of screening for a test compound that increases Na⁺ dependent fluid uptake by a pulmonary epithelial cell comprising contacting a pulmonary epithelial cell with the test compound in the presence of an excess of UTP, detecting Na⁺ dependent fluid uptake by the pulmonary epithelial cell, an increase in Na⁺ dependent fluid uptake as compared to a control indicating a test compound that increases Na⁺ dependent fluid uptake by a pulmonary epithelial cell.

48. (canceled)

49. (canceled)

50. The method of claim 47, further comprising removing the UTP and detecting reversibility of the increase in Na⁺ dependent fluid uptake.

51. A method of screening for a test compound that increases Na⁺ dependent fluid uptake by a cell comprising contacting the test compound with a cell that expresses a heterologous nucleic acid that encodes a pyrimidine synthesis gene, and detecting Na⁺ dependent fluid uptake by the cell, an increase in Na⁻ dependent fluid uptake as compared to a control level, indicating a test compound that increases Na⁺ dependent fluid uptake.

52. (canceled)

53. (canceled)

54. A method of screening for a test compound that increases Na⁺ dependent fluid uptake by a respiratory epithelial cell comprising infecting a H441 cell or cell line with respiratory syncytial virus, contacting the infected cell or cell line with a pyrimidine synthesis inhibitor, and measureing ion transport across the infected cell or cells of the infected cell line, an increase in ion transport as compared to a control level, indicating a test compound that increases Na⁺ dependent fluid uptake.

55. (canceled)

56. (canceled)
